(19) United States
(12) Patent Application Publication
(10) Pub. No.: US 2018/0369211 A1
(43) Pub. Date: Dec. 27, 2018

(54) METHODS AND COMPOSITIONS FOR TREATING NEURODEGENERATIVE DISORDERS

(71) Applicant: Lysosomal Therapeutics Inc.

(72) Inventors: Peter T. Lansbury, Brookline, MA (US); Renato T. Skerlj, West Newton, MA (US)

(21) Appl. No.: 15/739,149

(22) PCT Filed: Jun. 23, 2016
(86) PCT No.: PCT/US2016/039003

§ 371 Date: Dec. 21, 2017

(51) Int. Cl. A61K 31/423; A61K 31/513; A61P 25/16; A61P 25/28

(52) U.S. Cl. CPC A61K 31/423; A61P 25/28; A61P 25/16; A61K 31/513

(57) ABSTRACT

The invention relates to the treatment of neurodegenerative disorders with ceramidase inhibitor.

### CROSS-REFERENCE TO RELATED APPLICATIONS

This application claims the benefit of and priority to U.S. Provisional Patent Application No. 62/184,523, filed Jun. 25, 2015, the contents of which are hereby incorporated by reference.

### FIELD OF THE INVENTION

The invention relates generally to the methods and composition for treating neurodegenerative disorders, and more specifically the invention relates acid ceramidase inhibitors and their use in the treatment of neurodegenerative disorders.

### BACKGROUND

Neurodegenerative disorders often are associated with a reduction in the mass and/or volume of the brain, which may be due to the atrophy and/or death of brain cells, which are far more profound than those in a healthy subject that are attributable to aging. Neurodegenerative disorders can evolve gradually, after a long period of normal brain function, due to progressive degeneration (e.g., nerve cell dysfunction and death) of specific brain regions. Alternatively, neurodegenerative disorders can have a quick onset, such as those associated with trauma or toxins. The actual onset of brain degeneration may precede clinical expression by many years.

Examples of neurodegenerative disorders include, for example, Alzheimer's disease, Parkinson's disease, Huntington's disease, amyotrophic lateral sclerosis (ALS; also known as Lou Gehrig's disease or motor neuron disease), multiple sclerosis, and diffuse Lewy body disease. Once clinical expression occurs, the neurodegenerative disorder may be associated with impairment of motor function, for example, as observed in subjects with Parkinson's disease, Huntington's disease multiple sclerosis, or ALS. Alternatively or in addition, neurodegenerative disorders may be associated with cognitive impairment and/or the loss of cognitive function, for example, as observed in subjects with Alzheimer's disease.

Although significant effort has been made to develop treatments for these neurodegenerative disorders, there still remains a critical need for new approaches for treating neurodegenerative disorders.

### SUMMARY

The invention is based, in part, upon the fact that certain sphingosine-containing analogs accumulate to abnormal levels in the lysosomes or lysosomal compartments of cells of subjects with a neurodegenerative disorder, which can contribute to disease progression in those subjects. Given that acid ceramidase enzymes are involved in the conversion of ceramide-based substrates into sphingosine or sphingosine-containing analogs, acid ceramidase inhibitors may be used to treat a neurodegenerative disorder, for example, to slow down, stop, or reverse the development of the neurodegenerative disorder or ameliorate one or more symptoms of the neurodegenerative disorder.

In one aspect, the invention provides a method of treating a neurodegenerative disease in a subject in need thereof. The method comprises administering to the subject an acid ceramidase inhibitor in an amount effective to treat the disorder in the subject. The neurodegenerative disorder can include, for example, Parkinson's disease, Alzheimer's Disease, Huntington's Disease, amyotrophic lateral sclerosis, multiple sclerosis, diffuse Lewy body disease, multisystem atrophy, frontotemporal dementia, or progressive supranuclear palsy.

The acid ceramidase inhibitor can be administered to the subject so as to prevent the accumulation of sphingosine or a sphingosine-containing analog to a level found in subjects with a given neurodegenerative disorder when compared to subjects without the disorder. In other words, the acid ceramidase prevents the accumulation of a target sphingosine or sphingosine-containing analog to a predetermined threshold concentration (for example, a median concentration determined by clinical analyses) found in subjects with the neurodegenerative disorder relative to subjects without the disorder (i.e., less than the predetermined threshold concentration).

It is contemplated that a variety of acid ceramidase inhibitors, either alone or in combination with other agents, may be useful in the treatment of the neurodegenerative disorder. When administered, the acid ceramidase inhibitor prevents the accumulation of unwanted sphingosine or sphingosine-containing analogs, which are associated with the phenotype of the neurodegenerative disorder. An exemplary acid ceramidase inhibitor useful in treating one or more of the neurodegenerative disorders described herein can be a compound of Formula I or Formula I-1 below, for example

### (a) Formula I:

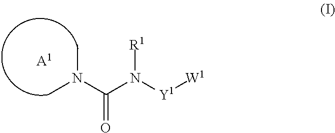

or a pharmaceutically acceptable salt thereof wherein:

A¹ is a cyclic group selected from 5-6 membered heterocyclyl, 5-6 membered heteroaryl, and bicyclic heterocyclyl, each of which is substituted by 1, 2, 3, or 4 occurrences of R²;

R¹ represents independently for each occurrence hydrogen, C₁₋₄alkyl, —C₁₋₄alkyl-phenyl, —CO₂—C₁₋₆alkyl, —C(O)—NH₂, —C(O)—NH—C₁₋₆alkyl, or —C(O)—N(C₁₋₆alkyl)₂;

R² represents independently for each occurrence R¹, C₁₋₄alkyl, C₁₋₄haloalkyl, C₁₋₄alkoxy, halogen, hydroxyl, oxo, cyano, nitro, azido, —N(R¹)₂, —C(O)—C₁₋₄alkyl, —C(O)-phenyl, —CO₂—R¹, —C(O)—NH₂, —C(O)—NH—C₁₋₆alkyl, —C(O)—N(C₁₋₆alkyl)₂, —O—C(O)—NH₂, —O—C(O)—NH—C₁₋₆alkyl, —O—C(O)N(C₁₋₆alkyl)₂, —C₁₋₄alkyl-phenyl, C₃₋₁₀cycloalkyl, C₃₋₁₀heterocyclyl, 6-10 membered aryl, 6-10 membered heteroaryl, —C₁₋₄alkylene-C₃₋₁₀cycloalkyl, —C₁₋₄alkylene-C₃₋₁₀heterocyclyl, —(C₁₋₄alkylene)-6-10 membered aryl, or —(C₁₋₄alkylene)-6-10 membered heteroaryl;

Y¹ represents:
C₁₋₁₈alkylene, C₂₋₁₈alkenylene, or C₂₋₁₈alkynylene;
C₃₋₁₀cycloalkylene, 3-10 membered heterocyclylene, 6-10 membered arylene, or 6-10 membered heteroarylene, each of which is substituted by 0, 1, 2, or 3 occurrences of C₁₋₄alkyl; or
R¹ and Y¹ together with the nitrogen to which they are attached form a 3-10 membered heterocyclylene; and

W¹ represents:
hydrogen; or
C₃₋₁₀cycloalkylene, C₃₋₁₀heterocyclylene, 6-10 membered arylene, or 6-10 membered heteroarylene; or

### (b) Formula I-1:

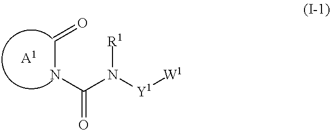

or a pharmaceutically acceptable salt thereof, wherein:

A¹ is a cyclic group selected from 5-6 membered heterocyclyl, 5-6 membered heteroaryl, and bicyclic heterocyclyl, each of which is substituted by 1, 2, or 3 occurrences of R²;

R¹ represents independently for each occurrence hydrogen, C₁₋₄alkyl, —C₁₋₄alkyl-phenyl, —CO₂—C₁₋₆alkyl, —C(O)—NH₂, —C(O)—NH—C₁₋₆alkyl, or —C(O)—N(C₁₋₆alkyl)₂;

R² represents independently for each occurrence R¹, C₁₋₄alkyl, C₁₋₄haloalkyl, C₁₋₄alkoxy, halogen, hydroxyl, oxo, cyano, nitro, azido, —N(R¹)₂, —C(O)—C₁₋₄alkyl, —C(O)-phenyl, —CO₂—R¹, —C(O)—NH₂, —C(O)NH—C₁₋₆alkyl, —C(O)—N(C₁₋₆alkyl)₂, —O—C(O)—NH₂, —O—C(O)—NH—C₁₋₆alkyl, —O—C(O)—N(C₁₋₆alkyl)₂, —C₁₋₄alkyl-phenyl, C₃₋₁₀cycloalkyl, C₃₋₁₀heterocyclyl, 6-10 membered aryl, 6-10 membered heteroaryl, —C₁₋₄alkylene-C₃₋₁₀cycloalkyl, —C₁₋₄alkylene-C₃₋₁₀heterocyclyl, —C₁₋₄alkylene-6-10 membered aryl, or —C₁₋₄alkylene-6-10 membered heteroaryl;

Y¹ represents:
C₁₋₁₈alkylene, C₂₋₁₈alkenylene, or C₂₋₁₈alkynylene;
C₃₋₁₀cycloalkylene, 3-10 membered, 6-10 membered arylene, or 6-10 membered heteroarylene, each of which is substituted by 0, 1, 2, or 3 occurrences of C₁₋₄alkyl; or
R¹ and Y¹ together with the nitrogen to which they are attached form a 3-10 membered heterocyclylene; and

W¹ represents:
hydrogen; or
C₃₋₁₀cycloalkylene, C₃₋₁₀heterocyclylene, 6-10 membered arylene, or 6-10 membered heteroarylene.

In certain embodiments, the acid ceramidase inhibitor is selected from the group consisting of:

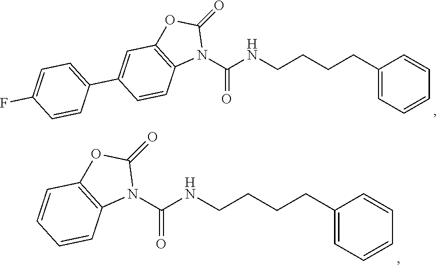

and pharmaceutically acceptable salts thereof.

In certain embodiments, the acid ceramidase inhibitor is a uracil analog, for example, a 5-fluorouracil analog. In one embodiment, the acid ceramidase inhibitor is 1-hexylcarbamoyl-5-fluorouracil, also known as Carmofur, whose chemical structure shown below:

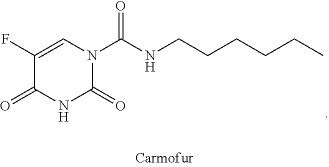 [SMILES: CCCCCCNC(=O)n1cc(F)c(=O)[nH]c1=O]

It is contemplated that, when the acid ceramidase inhibitor is a 5-florouracil analog, such as Carmofur, the 5-fluorouracil analog is administered at a concentration sufficient to inhibit acid ceramidase without substantially inhibiting thymidylate synthase.

In certain embodiments, the acid ceramidase inhibitor is administered at a concentration in the range from 0.01-200 mg/kg (for example, less than 20 mg/kg). In certain other embodiments, the acid ceramidase inhibitor is administered to a subject in the range from 0.01-200 mg/kg and at a dose sufficient to inhibit or reduce acid ceramidase activity but without substantially inhibiting (e.g., inhibiting less than 50%, 40%, 30%, 20%, 10%, or 5%) thymidylate synthase activity as determined in a cell or tissue sample using in vitro assays for measuring acid ceramidase activity, for example, as described in Bedia et al. (2010) J. LIPID RES. 51: 3542-3547, and thymidylate synthase activity, for example, as described in Pluim et al. (2013) ANAL. BIOANAL. CHEM. 405:2495-2503; Smith et al. (1967) J. BIOL. CHEM. 242: 109-113; Yalowich & Kalman (1985) BIOCHEM. PHARMACOL. 34: 2319-2324; and Cox & Harmenberg (1992) J. BIOCHEM. BIOPHYS. METHODS 25: 17-23.

The acid ceramidase inhibitors can be administered either alone or in combination with other agents for treating the neurodegenerative disorder.

These and other aspects and embodiments will be apparent from the following FIGURES, detailed description, and claims.

### BRIEF DESCRIPTION OF THE DRAWINGS

FIG. 1 is a schematic illustration showing various pathways of lysosomal sphingolipid degradation, including various lysosomal enzymes and their substrates, some of which are involved in certain lysosomal storage disorders and/or neurodegenerative disorders.

### DETAILED DESCRIPTION

The invention is based, in part, upon the observation that sphingosine-containing analogs (for example, glucosylsphingosine, galactosphingosine, lactosylsphingosine, GB3-sphingosine, and GM2-sphingosine) may accumulate in cells of subjects with certain neurodegenerative disorders and that the accumulation of these sphingosine-containing analogs may contribute to the disease phenotype. Given that these sphingosine-containing analogs are produced by acid ceramidase enzymes in the lysosomal compartments of cells, the accumulation of the sphingosine-containing analogs to detrimental levels can be prevented by the use of an effective amount of one or more inhibitors of acid ceramidase activity.

### I. Definitions

To facilitate an understanding of the present invention, a number of terms and phrases are defined below.

The terms “a” and “an” as used herein mean “one or more” and include the plural unless the context is inappropriate.

Acid ceramidase is understood to mean an amidase enzyme that catalyzes the conversion ceramide or ceramide-based substrates to their respective sphingosine or sphingosine-containing analogs via a deacylation reaction.

The term “acid ceramidase inhibitor” is understood to mean a compound that preferentially reduces the activity of an acid ceremidase enzyme relative to other mammalian enzymes, for example, other enzymes present in lysosomes of mammalian cells.

A “lysosomal storage disorder or LSD” is understood to mean a disorder associated with a deficiency in a glycosphingolipid hydrolase activity (either by a complete or partial loss of activity) in the lysosomes of mammalian cells. As a result of the deficiency of the glycosphingolipid hydrolase activity, the cells accumulate the substrate of the particular hydrolase. Exemplary, lysosomal storage disorders include, Gaucher's disease, Krabbe's disease, Fabry's disease, Tay-Sachs disease, Sandhoff Variant A, B disease, Niemann-Pick types A and B.

A “neurodegenerative disorder” is understood to mean a disorder in which neurons, over time, become dysfunctional and die. The pathology of neurodegenerative disorder typically is associated with protein aggregation and/or mitochondrial dysfunction. Exemplary neurodegenerative disorders include Parkinson's disease, Alzheimer's Disease, Huntington's Disease, amyotrophic lateral sclerosis, multiple sclerosis, diffuse Lewy body disease, multisystem atrophy, frontotemporal dementia, or progressive supranuclear palsy.

The term “alkyl” as used herein refers to a saturated straight or branched hydrocarbon, such as a straight or branched group of 1-12, 1-10, or 1-6 carbon atoms, referred to herein as C₁-C₁₂alkyl, C₁-C₁₀alkyl, and C₁-C₆alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “alkylene” refers to a diradical of an alkyl group. An exemplary alkylene group is —CH₂CH—.

The term “haloalkyl” refers to an alkyl group that is substituted with at least one halogen. For example, —CH₂F, —CHF₂, —CF₃, —CH₂CF₃, —CF₂CF₃, and the like.

The term “heteroalkyl” as used herein refers to an “alkyl” group in which at least one carbon atom has been replaced with a heteroatom (e.g., an O, N, or S atom). The heteroalkyl may be, for example, an —O—C₁-C₁₀alkyl group, an —C₁-C₆alkylene-O—C₁-C₆alkyl group, or a C₁-C₆ alkylene-OH group. In certain embodiments, the “heteroalkyl” may be 2-8 membered heteroalkyl, indicating that the heteroalkyl contains from 2 to 8 atoms selected from the group consisting of carbon, oxygen, nitrogen, and sulfur. In yet other embodiments, the heteroalkyl may be a 2-6 membered, 4-8 membered, or a 5-8 membered heteroalkyl group (which may contain for example 1 or 2 heteroatoms selected from the group oxygen and nitrogen). One type of heteroalkyl group is an “alkoxyl” group.

The term “alkenyl” as used herein refers to an unsaturated straight or branched hydrocarbon having at least one carbon-carbon double bond, such as a straight or branched group of 2-12, 2-10, or 2-6 carbon atoms, referred to herein as C₂-C₁₂alkenyl, C₂-C₁₀alkenyl, and C₂-C₆alkenyl, respectively. Exemplary alkenyl groups include vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl, and the like.

The term “alkynyl” as used herein refers to an unsaturated straight or branched hydrocarbon having at least one carbon-carbon triple bond, such as a straight or branched group of 2-12, 2-10, or 2-6 carbon atoms, referred to herein as C₂-C₁₂alkynyl, C₂-C₁₀alkynyl, and C₂-C₆alkynyl, respectively. Exemplary alkynyl groups include ethynyl, prop-1-yn-1-yl, and but-1-yn-1-yl.

The term “cycloalkyl” refers to a monovalent cyclic, bicyclic, or bridged cyclic (e.g., adamantyl) hydrocarbon group of 3-12, 3-8, 4-8, or 4-6 carbons, referred to herein, e.g., as “C₄₋₈cycloalkyl.” Cycloalkyl may contain one or more double bonds but does not have a completely conjugated pi-electron system. Exemplary cycloalkyl groups include, but are not limited to, cyclohexanes, cyclopentanes, cyclobutanes and cyclopropanes. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, nitro, azido, carbamate, carbonate, carboxy, cyano, cycloalkyl, ester, ether, formyl, halogen, haloalkyl, heteroaryl, heterocyclyl, hydroxyl, Imino, ketone, nitro, phosphate, phosphonato, phosphinato, sulfate, sulfide, sulfonamido, sulfonyl or thiocarbonyl. Cycloalkyl groups can be fused to other cycloalkyl, aryl, or heterocyclyl groups. In certain embodiments, the cycloalkyl group is not substituted, i.e., it is unsubstituted.

The term “cycloalkylene” refers to a diradical of an cycloalkyl group. An exemplary cycloalkylene group is

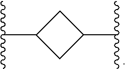

The term “cycloalkenyl” as used herein refers to a monovalent unsaturated cyclic, bicyclic, or bridged cyclic (e.g., adamantyl) hydrocarbon group of 3-12, 3-8, 4-8, or 4-6 carbons containing one carbon-carbon double bond, referred to herein, e.g., as “C₄₋₈cycloalkenyl,” derived from a cycloalkane. Exemplary cycloalkenyl groups include, but are not limited to, cyclohexenes, cyclopentenes, and cyclobutenes. Unless specified otherwise, cycloalkenyl groups are optionally substituted at one or more ring positions with, for example, alkanoyl, alkoxy, alkyl, alkenyl, alkynyl, amido, amidino, amino, aryl, arylalkyl, nitro, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. In certain embodiments, the cycloalkenyl group is not substituted, i.e., it is unsubstituted.

The term “aryl” is art-recognized and refers to a carbocyclic aromatic group. Representative aryl groups include phenyl, naphthyl, anthracenyl, and the like. The term “aryl” includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. Unless specified otherwise, the aromatic ring may be substituted at one or more ring positions with, for example, halogen, nitro, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the aromatic ring is substituted at one or more ring positions with halogen, alkyl, hydroxyl, or alkoxyl. In certain other embodiments, the aromatic ring is not substituted, i.e., it is unsubstituted. In certain embodiments, the aryl group is a 6-10 membered ring structure.

The term “aralkyl” refers to an alkyl group substituted with an aryl group.

The term “bicyclic carbocyclyl that is partially unsaturated” refers to a bicyclic carbocyclic group containing at least one double bond between ring atoms and at least one ring in the bicyclic carbocyclic group is not aromatic. Representative examples of a bicyclic carbocyclyl that is partially unsaturated include, for example:

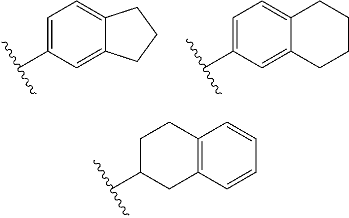 [SMILES: *c1ccc2c(c1)CCC2 ; *c1ccc2c(c1)CCCC2 ; *C1CCc2ccccc2C1]

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

The terms “heterocyclyl” and “heterocyclic group” we art-recognized and refer to saturated, partially unsaturated, or aromatic 3- to 10-membered ring structures, alternatively 3- to 7-membered rings, whose ring structures include one to four heteroatoms, such as nitrogen, oxygen, and sulfur. The number of ring atoms in the heterocyclyl group can be specified using Cₓ-Cₓ nomenclature where x is an integer specifying the number of ring atoms. For example, a C₃-C₇heterocyclyl group refers to a saturated or partially unsaturated 3- to 7-membered ring structure containing one to four heteroatoms, such as nitrogen, oxygen, and sulfur. The designation “C₃-C₇” indicates that the heterocyclic ring contains a total of from 3 to 7 ring atoms, inclusive of any heteroatoms that occupy a ring atom position. One example of a C₃heterocyclyl is aziridinyl. Heterocycles may also be mono-, bi-, or other multi-cyclic ring systems. A heterocycle may be fused to one or more aryl, partially unsaturated, or saturated rings. Heterocyclyl groups include, for example, biotinyl, chromenyl, dihydrofuryl, dihydroindolyl, dihydropyranyl, dihydrothienyl, dithiazolyl, homopiperidinyl, imidazolidinyl, isoquinolyl, isothiazolidinyl, isooxazolidinyl, morpholinyl, oxolanyl, oxazolidinyl, phenoxanthenyl, piperazinyl, piperidinyl, pyranyl, pyrazolidinyl, pyrazolinyl, pyridyl (i.e., pyridinyl), pyrimidinyl, pyrrolidinyl, pyrrolidin-2-onyl, pyrrolinyl, tetrahydrofuryl, tetrahydroisoquinolyl, tetrahydropyranyl, tetrahydroquinolyl, thiazolidinyl, thiolanyl, thiomorpholinyl, thiopyranyl, xanthenyl, lactones, lactams such as azetidinones and pyrrolidinones, sultams, sultones, and the like. Heterocyclyl groups also include, for example, furanyl, pyrrolyl, thiophenyl, pyrazolyl, oxazolyl, thiazolyl, tetrahydropyrimidinyl, pyrazinyl, dihydroisooxazolyl, isooxazolyl, isothiazolyl, imidazolyl, oxadiazolyl, thiadiazolyl, imidazolinyl, imidazolidinyl, oxazolinyl, pyrazolinyl, thiazolinyl, triazolinyl, dihydrobenzooxazolyl, dihydrobenzoisoxazole, dihydrobenzothiazolyl, dihydrooxazolopyridinyl, dihydroimidazopyridinyl, dihydropyrazolopyridinyl, dihydroindazolyl, dihydrobenzoisothiazolyl, dihydroisothiazolopyridine, indazolyl, benzotriazolyl, triazolopyridine, and the like. Unless specified otherwise, the heterocyclic ring is optionally substituted at one or more positions with substituents such as alkanoyl, alkoxy, alkyl, alkenyl, alkynyl, amido, amidino, amino, aryl, arylalkyl, nitro, azido, carbamate, carbonate, carboxy, cyano, cycloalkyl, ester, ether, formyl, halogen, haloalkyl, heteroaryl, heterocyclyl, hydroxyl, imino, ketone, nitro, oxo, phosphate, phosphonato, phosphinato, sulfate, sulfide, sulfonamido, sulfonyl and thiocarbonyl. In certain embodiments, the heterocyclyl group is not substituted, i.e., it is unsubstituted.

The term “bicyclic heterocyclyl” refers to a heterocyclyl group that contains two rings that are fused together. Representative examples of a bicyclic heterocyclyl include, for example:

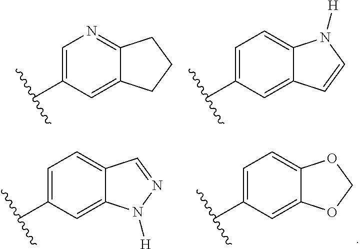 [SMILES: *c1cnc2c(c1)CCC2 ; *c1ccc2[nH]ccc2c1 ; *c1ccc2cn[nH]c2c1 ; *c1ccc2c(c1)OCO2]

In certain embodiments, the bicyclic heterocyclyl is an carbocyclic ring fused to partially unsaturated heterocyclic ring, that together form a bicyclic ring structure having 8-10 ring atoms (e.g., where there are 1, 2, 3, or 4 heteroatoms selected from the group consisting of nitrogen, oxygen, and sulfur).

The term “heterocycloalkyl” is art-recognized and refers to a saturated heterocyclyl group as defined above. In certain embodiments, the “heterocycloalkyl” is a 3- to 10-membered ring structures, alternatively a 3- to 7-membered rings, whose ring structures include one to four heteroatoms, such as nitrogen, oxygen, and sulfur.

The term “heterocycloalkylene” refers to a diradical of a heterocycloalkyl group. An exemplary heterocycloalkylene group is

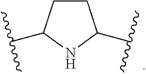

The heterocycloalkylene may contain, for example, 3-6 ring atom (i.e., a 3-6 membered heterocycloalkylene). In certain embodiments, the heterocycloalkylene is a 3-6 membered heterocycloalkylene containing 1, 2, or 3 three heteroatoms selected from the group consisting of oxygen, nitrogen, and sulfur.

The term “heteroaryl” is art-recognized and refers to aromatic groups that include at least one ring heteroatom. In certain instances, a heteroaryl group contains 1, 2, 3, or 4 ring heteroatoms. Representative examples of heteroaryl groups include pyrrolyl, furanyl, thiophenyl, imidazolyl, oxazolyl, thiazolyl, triazolyl, pyrazolyl, pyridinyl, pyrazinyl, pyridazinyl and pyrimidinyl, and the like. Unless specified otherwise, the heteroaryl ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “heteroaryl” also includes polycyclic ring systems having two or more rings in which two or more carbons are common to two adjoining rings (the rings are “fused rings”) wherein at least one of the rings is heteroaromatic, e.g., the other cyclic rings may be cycloalkyls, cycloalkenyls, cycloalkynyls, and/or aryls. In certain embodiments, the heteroaryl ring is substituted at one or more ring positions with halogen, alkyl, hydroxyl, or alkoxyl. In certain other embodiments, the heteroaryl ring is not substituted, i.e., it is unsubstituted. In certain embodiments, the heteroaryl group is a 5- to 10-membered ring structure, alternatively a 5- to 6-membered ring structure, whose ring structure includes 1, 2, 3, or 4 heteroatoms, such as nitrogen, oxygen, and sulfur.

The term “heteroaralkyl” refers to an alkyl group substituted with a heteroaryl group.

The terms “amine” and “amino” are art-recognized and refer to both unsubstituted and substituted amines, e.g., a moiety represented by the general formula —N(R⁵⁰)(R⁵¹), wherein R⁵⁰ and R⁵¹ each independently represent hydrogen, alkyl, cycloalkyl, heterocyclyl, alkenyl, aryl, aralkyl, or —(CH₂)ₘ—R⁶¹; or R⁵⁰ and R⁵¹, taken together with the N atom to which they are attached complete a heterocycle having from 4 to 8 atoms in the ring structure; R⁶¹ represents an aryl, a cycloalkyl, a cycloalkenyl, a heterocycle or a polycycle; and m is zero or an integer in the range of 1 to 8. In certain embodiments, R⁵⁰ and R⁵¹ each independently represent hydrogen, alkyl, alkenyl, or —(CH₂)ₘ—R⁶¹.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, —O—(CH₂)ₘ—R₆₁, where m and R₆₁ are described above.

The term “carbamate” as used herein refers to a radical of the form —R_{g}OC(O)N(Rₕ)—, —R_{g}OC(O)N(Rₕ)Rᵢ₋, or —OC(O)NRₕRᵢ, wherein R_{g}, Rₕ and Rᵢ are each independently alkoxy, aryloxy, alkyl, alkenyl, alkynyl, amide, amino, aryl, arylalkyl, carboxy, cyano, cycloalkyl, ester, ether, formyl, halogen, haloalkyl, heteroaryl, heterocyclyl, hydroxyl, ketone, nitro, sulfide, sulfonyl, or sulfonamide. Exemplary carbamates include arylcarbamates and heteroaryl carbamates, e.g., wherein at least one of R_{g}, Rₕ and Rᵢ are independently aryl or heteroaryl, such as phenyl and pyridinyl.

The term “carbonyl” as used herein refers to the radical —C(O)—.

The term “carboxamido” as used herein refers to the radical —C(O)NRR′, where R and R′ may be the same or different. R and R′ may be independently alkyl, aryl, arylalkyl, cycloalkyl, formyl, haloalkyl, heteroaryl, or heterocyclyl.

The term “carboxy” as used herein refers to the radical —COOH or its corresponding salts, e.g. —COONa, etc.

The term “amide” or “amido” as used herein refers to a radical of the form —RₐC(O)N(R_{b})—, —RₐC(O)N(R_{b})R_{c}—, —C(O)NR_{b}R_{c}, or —C(O)NH₂, wherein Rₐ, R_{b}, and R_{c} are each independently alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, or nitro. The amide can be attached to another group through the carbon, the nitrogen, R_{b}, R_{c}, or Rₐ. The amide also may be cyclic, for example R_{b} and R_{c}, Rₐ and R_{b}, or Rₐ and R_{c} may be joined to form a 3- to 12-membered ring such as a 3- to O-membered ring or a 5- to 6-membered ring.

The term “amidino” as used herein refers to a radical of the form —C(═NR)NR′R″ where R, R′, and R″ are each independently alkyl, alkenyl, alkynyl, amide, aryl, arylalkyl, cyano, cycloalkyl, haloalkyl, heteroaryl, heterocyclyl, hydroxyl, ketone, or nitro.

The term “alkanoyl” as used herein refers to a radical —O—CO-alkyl.

The term “oxo” is art-recognized and refers to a “═O” substituent. For example, a cyclopentane substituted with an oxo group is cyclopentanone.

The term “sulfonamide” or “sulfonamido” as used herein refers to a radical having the structure —N(Rᵣ)—S(O)₂—Rₛ— or —S(O)₂—N(Rᵣ)Rₛ, where Rᵣ, and Rₛ can be, for example, hydrogen, alkyl, aryl, cycloalkyl, and heterocyclyl. Exemplary sulfonamides include alkylsulfonamides (e.g., where Rₛ is alkyl), arylsulfonamides (e.g., where Rₛ is aryl), cycloalkyl sulfonamides (e.g., where Rₛ is cycloalkyl), and heterocyclyl sulfonamides (e.g., where Rₛ is heterocyclyl), etc.

The term “sulfonyl” as used herein refers to a radical having the structure RᵤSO₂—, where Rᵤ can be alkyl, aryl, cycloalkyl, and heterocyclyl, e.g., alkylsulfonyl. The term “alkylsulfonyl” as used herein refers to an alkyl group attached to a sulfonyl group.

The symbol “ [symbol] ” indicates a point of attachment.

Unless otherwise indicated, the term “substituted” as used herein means that one or more hydrogen atoms of the above mentioned groups are replaced with another atom or functional group including, by way of example, alkyl, substituted alkyl, aryl, substituted aryl, arylalkyl, alkoxy, cycloalkyloxy, aryloxy, arylalkyloxy, hydroxy, heteroaryl, heteroaryloxy, heterocyclyloxy, trifluoromethyl, trifluoromethoxy, carboxy, acyl, aroyl, heteroaroyl, halogen, nitro, cyano, alkoxycarbonyl, aryloxycarbonyl, aralkyloxycarbonyl, cycloalkyloxycarbonyl, heteroaryloxycarbonyl, acyloxy, alkylthio, arylthio, alkysulfinyl, arylsulfinyl, alkylsulfonyl, arylsulfonyl, —O-aroyl, —O-heteroaroyl, oxo (═O), —C(═O)—NRₕRₖ, and —NRₚR_{q} wherein each of Rₕ, Rₖ, Rₚ, and R_{q} independently represents hydrogen, unsubstituted or substituted alkyl, unsubstituted or substituted cycloalkyl, unsubstituted or substituted aryl, unsubstituted or substituted arylalkyl, unsubstituted or substituted heteroaryl, unsubstituted or substituted heterocyclyl, acyl, aroyl, heteroaroyl, and when Rₕ and Rₖ, or Rₚ and R_{q} are taken together with the nitrogen atom to which they are bound, the group —NRₕRₖ or the group NRₚR_{q} represent a heterocyclyl residue and wherein the terms alkyl, cycloalkyl, aryl, heteroaryl, heterocyclyl are as defined herein.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. It is understood that graphical depictions of chemical structures, e.g., generic chemical structures, encompass all stereoisomeric forms of the specified compounds, unless indicated otherwise.

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Further, enantiomers can be separated using supercritical fluid chromatographic (SFC) techniques described in the literature. Still further, stereoisomers can be obtained from stereomerically-pure intermediates, reagents, and catalysts by well-known asymmetric synthetic methods.

Geometric isomers can also exist in the compounds of the present invention. The symbol [symbol] denotes a bond that may be a single, double or triple bond as described herein. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “2” or “E” configuration wherein the terms “Z” and “E” are used in accordance with IUPAC standards. Unless otherwise specified, structures depicting double bonds encompass both the “E” and “Z” isomers.

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The invention also embraces isotopically labeled compounds of the invention which are identical to those recited herein, except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, H, ¹³C, ¹⁴C, ¹⁵N, ¹⁸O, ¹⁷O, ³¹P, ³²P, ³⁵, S, ¹⁸F, and ³⁶Cl, respectively.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the invention can generally be prepared by following procedures analogous to those disclosed in, e.g., the Examples herein by substituting an isotopically labeled reagent for a non-isotopically labeled reagent.

As used herein, the terms “subject” and “patient” refer to organisms to be treated by the methods of the present invention. Such organisms are preferably mammals (e.g., murines, simians, equines, bovines, porcines, canines, felines, and the like), and more preferably humans.

The term “effective amount” refers to the amount of a compound (e.g., a compound of the present invention) or composition containing the compound sufficient to effect beneficial or desired results material. The term “therapeutically effective amount” refers to the amount of a compound (e.g., a compound of the present invention) or composition containing the compound effective for producing some desired therapeutic effect in at least a sub-population of cells in either a subject with or at risk of developing a neurodegenerative disorder or an animal at a reasonable benefit/risk ratio applicable to any medical treatment. An effective amount or therapeutically effective amount can be administered in one or more administrations, applications or dosages and is not intended to be limited to a particular formulation or administration route.

The term “treating” refers any effect, e.g., lessening, reducing, modulating, ameliorating, reversing or eliminating, that results in the improvement of the condition, disease, disorder, and the like, or ameliorating a symptom thereof.

The term “pharmaceutical composition” refers to the combination of an active agent with a carrier, inert or active, making the composition especially suitable for diagnostic or therapeutic use in vivo or ex vivo.

The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Martin, Remington's Pharmaceutical Sciences, 15th Ed., Mack Publ. Co., Easton, Pa. [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene p-sulfuric, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄′ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The terms “parenteral administration” and “administered parenterally” as used herein means modes of administration other than enteral and topical administration, usually by injection, and includes, without limitation, intravenous, intramuscular, intraarterial intrathecal, intracapsular, intraorbital, intracardiac, intradermal, intraperitoneal, transtracheal, subcutaneous, subcuticular, intraarticular, subcapsular, suberachnoid, intraspinal and intrasternal injection and infusion.

Abbreviations as used herein include O-(7-azabenzotriazol-1-yl)-N,N′,N′-tetramethyluronlum hexafluoophosphate (HATU); diisopropylethylamine (DIPEA); dimethylformamide (DMF); methylene chloride (DCM); tert-butoxycarbonyl (Boc); tetrahydrofuran (THF); trifluoroacetic acid (TFA); N-methylmorpholine (NMM); triethylamine (TEA); Boc anhydride ((Boc)₂O); dimethylsulfoxide (DMSO); diisopropylethylamine (DIEA); N,N-Dimethylpyridin-4-amine (DMAP); flash column chromatography (FCC); and supercritical fluid chromatography (SFC).

Throughout the description, where compositions and kits are described as having. including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### II. Therapeutic Applications

The invention provides a method for treating a neurodegenerative disorder in a subject in need thereof. The method comprises administering to the subject an effective amount of an acid ceramidase inhibitor sufficient to treat the disorder in the subject. The following sections describes acid ceramidase inhibitors useful in the practice of the invention when administered, either alone or in combination with other agents, to the subject in need of such treatment. It is contemplated that this approach can be useful in treating a variety of neurodegenerative disorders, including, Parkinson's disease, Alzheimer's Disease, Huntington's Disease, amyotrophic lateral sclerosis, multiple sclerosis, diffuse Lewy body disease, multisystem atrophy, frontotemporal dementia, or progressive supranuclear palsy.

Neurodegenerative disorders often are associated with reduction in the mass and/or volume of the brain, which may be due to the atrophy and/or death of brain cells, which are far more profound than those in a healthy subject that are attributable to aging. Neurodegenerative disorders can evolve gradually, after a long period of normal brain function, due to progressive degeneration (e.g., nerve cell dysfunction and death) of specific brain regions. Alternatively, neurodegenerative disorders can have a quick onset, such as those associated with trauma or toxins. The actual onset of brain degeneration may precede clinical expression by many years.

Alzheimer's disease is a central nervous system (CNS) disorder that results in memory loss, unusual behavior, personality changes, and a decline in thinking abilities. These losses are related to the death of specific types of brain cells and the breakdown of connections and their supporting network (e.g., glial cells) between them. The earliest symptoms include loss of recent memory, faulty judgment, and changes in personality. Parkinson's disease is a CNS disorder that results in uncontrolled body movements, rigidity, tremor, and dyskinesia, and is associated with the death of brain cells in an area of the brain that produces dopamine. ALS (motor neuron disease) is a CNS disorder that attacks the motor neurons, components of the CNS that connect the brain to the skeletal muscles. Huntington's disease is another neurodegenerative disease that causes uncontrolled movements, loss of intellectual faculties, and emotional disturbance.

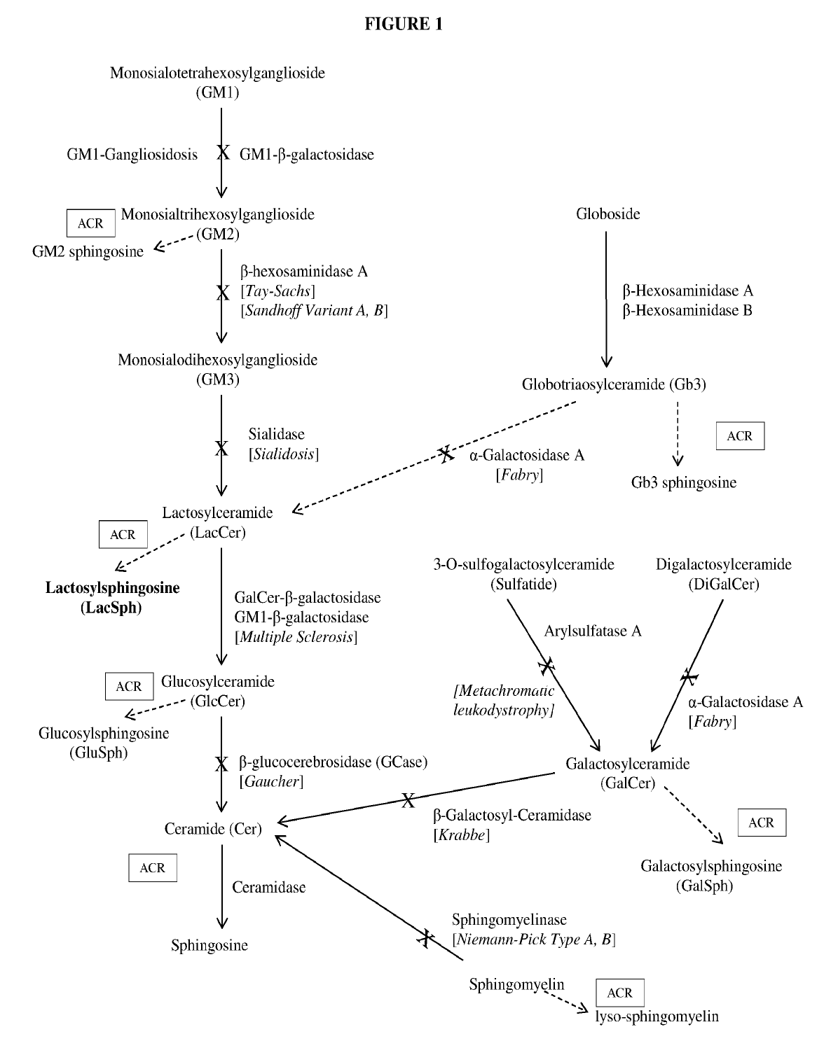

Certain neurodegenerative disorders appear to be associated with enzymatic activities implicated in certain lysosomal storage disorders. FIG. 1 shows certain enzymatic pathways involved in sphingolipid degradation in lysosomes. As shown in FIG. 1, defects in certain of the lysosomal enzymes may result in the development of various lysosomal storage disorders or LSDs. For example, Gaucher's disease is associated with defective β-glucocerebrosidase activity, Fabry's disease is associated with defective α-galactoside A activity, Krabbe's disease is associated with defective β-galactosyl-ceramidase activity, Niemann Pick disease types A and B is associated with defective sphingomyelinase activity, and Tay Sachs disease or Sandhoff Variant A, B is associated with defective β-hexosaminidase A activity.

In Gaucher's disease, for example, glucosylceramide (GlcCer) that accumulates as a result of deficient β-glucocerebrosidase activity can be converted into glucosylsphingosine (GluSph) via an acid ceramidase enzyme (a glycosylceramide to glycosylsphingosine converting enzyme). As a result, the accumulation of glucosylceramide (caused by defective β-glucocerebrosidase) may result in an accumulation of glucosylsphingosine, which is involved in disease progression in subjects with Gaucher's disease. Given that the conversion of glucosylceramide to glucosphingosine is catalyzed by an acid ceramidase enzyme, the administration of an acid ceramidase inhibitor can prevent the accumulation of glucosphingosine to a concentration or level within the lysosomal compartment that is toxic or otherwise detrimental to the cells of the subject. As a result, administration of an acid ceramidase inhibitor can reduce the accumulation of glucosphingosine thereby treating Gaucher's disease, which includes ameliorating a symptom associated with Gaucher's disease.

Similarly, in the case of Niemamnn-Pck type A or B, defective sphingomyelinase results in the accumulation of sphingomyelin, which in turn can be converted into lyso-sphinomyelin via an acid ceramidase (a sphingomyelin to lyso-sphingomyelin converting enzyme). Given the accumulation of lyso-sphingomyelin to a concentration or level within the lysosomal compartment that is toxic or otherwise detrimental to the cells in subjects with Niemann-Pick Type A or B, the administration of an acid ceramidase inhibitor can reduce the accumulation of lyso-sphingomyelin thereby treating Niemann-Pick type A or B, which includes ameliorating a symptom associated with Niemann-Pick type A or B.

It has been observed that subjects with certain mutant alleles in genes encoding β-glucocerebrosidase activity (the GBA gene; Aharon-Peretz (2004) NEW. ENG. J. MED. 351: 1972-1977; Gan-Or el al. (2008) NEUROLOGY 70:2277-2283; Gan-Or el al. (2015) NEUROLOGY 3:880-887) and sphinomyelinase activity (the SMPD1 gene, Gan-Or et al. (2013) NEUROLOGY 80:1606-1610) have been associated with, and identified as a risk factor for, Parkinson's Disease. As a result defects with, or deficiencies in the activities of these enzymes, as in the case of Gaucher's disease and Niemann Pick types A and B, may result in the accumulation of glucosylceramide and sphingomyelin, which can then be converted to glucosylsphingosine or lyso-sphingomyelin, respectively, via acid ceramidase activity. The accumulation of glucosylsphingosine or lyso-sphingomyelin may thus be implicated in the development of Parkinson's disease. It is contemplated that the administration of an acid ceramidase inhibitor, which slows down, stops or reverses the accumulation of glucosylsphingosine and/or lyso-sphingomyelin can be used to treat Parkinson's Disease. For example, an acid ceramidase inhibitor can be used to improve motor and/or memory impairments symptomatic of Parkinson's disease.

Tay-Sachs disease and Sandhoff disease are glycolipid storage disease where the GM2 ganglioside substrate for β-hexosaminidase accumulates in the nervous system. Although it has been contemplated that GM2 ganglioside can trigger acute neurodegeneration, the inventors note that GM2 ganglioside, which accumulates in these LSDs, is also converted to GM2-sphingosine via acid ceramidase activity. Elevated GM2-sphingosine may also be associated with triggering acute neurodegeneration. As a result, it is contemplated that the administration of an acid ceramidase inhibitor, which slows down, stops or reverses the accumulation of GM2-sphingosine can be used to treat acute neurodegeneration in a subject.

Similarly, it has been observed that lactosylceramide (LacCer) is upregulated in the central nervous system of mice during chronic experimental autoimmune encephalomyelitis (EAE), a model of multiple sclerosis (Lior et al. (2014) NATURE MEDICINE 20:1147-1156.). It is contemplated that the increase in LacCer may also result in the an increase in lactosylsphingosine (LacSph) via conversion by an acid ceramidase (a lactosylceramide to lactosylsphingosine converting enzyme). Given the accumulation of lactosylsphingosine to a toxic or otherwise detrimental level or concentration in the lysosomal compartments of cells in subjects with multiple sclerosis, the administration of an acid ceramidase inhibitor may reduce the accumulation of lactosylsphingosine thereby treating multiple sclerosis, which includes ameliorating a symptom associated with multiple sclerosis.

It has been observed that the level and activity of acid ceramidase can be elevated in subjects with Alzheimer's disease (Huang et al. (2004) EUROPEAN J. NEUROSCI. 20:3489-3497. Given that the accumulation of sphingosine or sphingosine analogs to a toxic or otherwise detrimental level or concentration in the lysosomal compartments of cells in subjects with Alzheimer's disease, the administration of an acid ceramidase inhibitor can reduce the accumulation of the sphingosine or sphingosine analogs thereby treating Alzheimer's disease, which includes ameliorating a symptom associated with Alzheimer's disease.

Furthermore, given that a number of the foregoing neurodegenerative disorders, for example, Alzheimer's disease, are associated with a level of cognitive impairment and/or some decrease or loss of cognitive function, it is contemplated that the administration of an effective of an acid ceramidase inhibitor to a subject in need thereof may be reduce, stabilize, or reverse cognitive impairment and/or the loss of cognitive function. Cognitive function generally refers to the mental processes by which one becomes aware of, perceives, or comprehends ideas. Cognitive function involves all aspects of perception, thinking, learning, reasoning, memory, awareness, and capacity for judgment. Cognitive impairment generally refers to conditions or symptoms involving problems with thought processes. This may manifest itself in one or more symptoms indicating a decrease in cognitive function, such as impairment or decrease of higher reasoning skills, forgetfulness, impairments to memory, learning disabilities, concentration difficulties, decreased intelligence, and other reductions in mental functions.

Cognitive function and cognitive impairment may be readily evaluated using tests well known in the art. Performance in these tests can be compared over time to determine whether a treated subject is improving or whether further decline has stopped or slowed, relative to the previous rate of decline of that patient or compared to an average rate of decline. Tests of cognitive function, including memory and learning for evaluating hum patients are well known in the art and regularly used to evaluate and monitor subjects having or suspected of having cognitive disorders such as Alzheimer's disease including the clock-drawing test (Agrell & Dehlin (1998) AGE & AGING 27:399-403). Even in healthy individuals, these and other standard tests of cognitive function can be readily used to evaluate beneficial affects over time.

### III. Acid Ceramidase Inhibitors

It is contemplated that a variety of acid ceramidase inhibitors can be used in the methods described herein.

It is contemplated that a variety of acid ceramidase inhibitors can be used in the practice of the invention. Exemplary acid ceramidase inhibitors can be tested for activity using a variety of in vitro assays known in the art, for example, as described in Bedia et al. (2010) supra. An exemplary assay may use a fluorogenic substrate as shown in Formula II

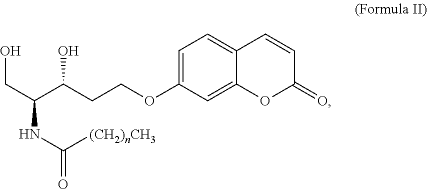

where different fatty acid chain lengths as denoted by integer n, which can be, for example, 6, 8, 10, 12, 14, 16, or 18. Exemplary fluorogenic analogs of Formula II include Rbm 14-10, Rbm 14-12, Rbm 14-14, and Rbm 14-16, where n can be 8, 10, 12, or 14, respectively, where Rbm 14-12 is preferred (Formula II, where n=8) (see, Bedla et al. (2010) supra). It is contemplated that exemplary acid ceramidase inhibitors reduce acid ceramidase activity by at least 20%, 30%, 40%, 50%, 60%, 70%, 80%, 90% or 95% in the assay set forth in Bedia et al. (2010) supra using the fluorogenic Rbm 14-12 substrate.

Exemplary acid ceramidase inhibitors are described in Realini et al. (2013) SCIENTIFIC REPORTS, 3:1035; Saied & 696,246, Arenz (2014) CELLULAR PHYSIOLOGY AND BIOCHEMISTRY, 34:197-212; Pizzirani et al. (2015) ANGEWANDTE CHEMIE INT. ED., 54:485-489; Poupeert et al. (2005) CURR. MED. CHEM., 12:877-885; Pizzirani et al. (2013) J. MED. CHEM., 56:3518-3530; Sun et al. (2013) BIOORG. MED. CHEM., 21:7724-7734; Goodman et al. (2009) BIOORG. MED. CHEM. LETT., 19:27-30; O'Connell et al. (2012) BIOORG. MED. CHEM. LETT., 22:1397-1401; U.S. Pat. No. 7,696,246, U.S. Pat. No. 6,964,973, U.S. Pat. No. 7,709,513; U.S. Pat. No. 7,846,943; WO 2006/131231; WO 2006/131232; WO 2006/131233; WO 2007/110215; WO 2007/110216; WO 2006/111321; WO 2007/042178; WO 2007/045393; WO 2007/045392; WO 2008/122352; WO 2008/122357; WO 2011/157827; WO 2004/094394; WO 2004/093872; WO 2004/094393; WO 2009/141627; WO 2013/151877; WO 2013/151923; WO 2013/151877; WO 2013/048928; WO 2013/048930; WO 2013/048942; WO 2013/048982; WO 2013/049096; WO 2013/049104; WO 2013/178576; WO 2014/042939; WO 2014/015088; WO 2014/011461; WO 2009/123164; WO 2009/133834; WO 2011/074560; WO 2012/081563; WO 2012/173099; and US 2014/0011799; each of which is hereby incorporated by reference in its entirety for all purposes.

In one embodiment, an exemplary acid ceramidase inhibitor is a compound of Formula I:

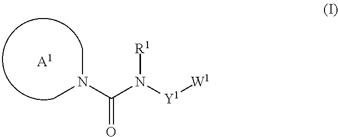

or a pharmaceutically acceptable salt thereof, wherein:

A¹ is a cyclic group selected from 5-6 membered heterocyclyl, 5-6 membered heteroaryl, and bicyclic heterocyclyl, each of which is substituted by 1, 2, 3, or 4 occurrences of R²;

R¹ represents independently for each occurrence hydrogen, C₁₋₄alkyl, —C₁₋₄alkyl-phenyl, —CO₂—C₁₋₆alkyl, —C(O)—NH₂, —C(O)—NH—C₁₋₆alkyl, or —C(O)—N(C₁₋₆alkyl)₂;

R² represents independently for each occurrence R¹, C₁₋₄alkyl, C₁₋₄haloalkyl, C₁₋₄alkoxy, halogen, hydroxyl, oxo, cyano, nitro, azido, —N(R¹)₂, —C(O)—C₁₋₄alkyl, —C(O)-phenyl, —CO₂—R¹, —C(O)—NH₂, —C(O)—NH—C₁₋₆alkyl, —C(O)—N(C₁₋₆alkyl)₂, —O—C(O)—NH₂, —O—C(O)—NH—C₁₋₆alkyl, —O—C(O)—N(C₁₋₆alkyl)₂, —C₁₋₄alkyl-phenyl, C₃₋₁₀cycloalkyl, C₃₋₁₀heterocyclyl, 6-10 membered aryl, 6-10 membered heteroaryl, —C₁₋₄alkylene-C₃₋₁₀cycloalkyl, —C₁₋₄alkylene-C₃₋₁₀heterocyclyl, —(C₁₋₄alkylene)-6-10 membered aryl, or —(C₁₋₄alkylene)-6-10 membered heteroaryl;

Y¹ represents:
C₁₋₁₈alkylene, C₂₋₁₈alkenylene, or C₂₋₁₈alkynylene;
C₃₋₁₀cycloalkylene, 3-10 membered heterocyclylene, 6-10 membered arylene, or 6-10 membered heteroarylene, each of which is substituted by 0, 1, 2, or 3 occurrences of C₁₋₄alkyl; or
R¹ and Y¹ together with the nitrogen to which they are attached form a 3-10 membered heterocyclylene; and

W¹ represents:
hydrogen; or
C₃₋₁₀cycloalkylene, C₃₋₁₀heterocyclylene, 6-10 membered arylene, or 6-10 membered heteroarylene.

Definitions of the variables in Formula I above encompass multiple chemical groups. The application contemplates embodiments where, for example, i) the definition of a variable is a single chemical group selected from those chemical groups set forth above, ii) the definition is a collection of two or more of the chemical groups selected from those set forth above, and ill) the compound is defined by a combination of variables in which the variables are defined by (i) or (ii), e.g., such as where Y¹ is C₁₋₁₈alkylene, W¹ is 6-10 membered arylene, and A¹ is bicyclic heterocyclyl.

In certain embodiments, R¹ represents hydrogen.

In certain embodiments, R² represents independently for each occurrence hydrogen, C₁₋₄alkyl, —C₁₋₄-phenyl, phenyl, halophenyl, —C(O)—C₁₋₄alkyl, methyl, isopropyl, fluoro, chloro, bromo, C₁₋₄haloalkyl, or trifluoromethyl.

In certain embodiments, Y¹ is C₁₋₁₈alkylene. For example, in certain embodiments, Y¹ may be C₁₋₆alkylene, C₁₋₄alkylene, methylene, ethylene, propylene, butylene, pentylene, hexylene, heptylene, octylene, or nonylene, decylene, undecylene, or dodecylene. In certain embodiments, Y¹ is 6-10 membered arylene. For example, in certain embodiments, Y¹ may be indanylene or tetralinylene.

In certain embodiments, W¹ is C₃₋₁₀cycloalkylene, C₃₋₁₀heterocyclylene, 6-10 membered arylene, or 6-10 membered heteroarylene, each of which may be substituted with one, two, or three occurrences of C₁₋₆alkyl or C₁₋₆alkoxy. In certain embodiments, W¹ is hydrogen, phenyl, methylphenyl, dimethylphenyl, cyclohexyl, methoxyphenyl, dimethoxyphenyl, or trimethoxyphenyl. In certain embodiments, W¹ is 6-10 membered arylene. For example, in certain embodiments, W¹ may be indanylene or tetralinylene.

In certain embodiments, A¹ is furanyl, pyrrolyl, thiophenyl, pyrazolyl, oxazolyl, thiazolyl, pyridinyl, pyrimidinyl, tetrahydropyrimidinyl, pyrazinyl, dihydroisooxazolyl, isooxazolyl, isothiazolyl, Imidazolyl, oxadiazolyl, thiadiazolyl, imidazolinyl, imidazolidinyl, oxazolinyl, pyrazolinyl, thiazolinyl, triazolinyl, dihydrobenzooxazolyl, dihydrobenzoisoxazole, dihydrobenzothiazolyl, dihydrooxazolopyridinyl, dihydroimidazopyridinyl, dihydropyrazolopyridinyl, dihydroindazolyl, dihydrobeniisothiazolyl, dihydroisothiazolopyridine, indazolyl, benzotriazolyl, or triazolopyridine. In certain embodiments, A¹ is furanyl, pyrrolyl, thiophenyl, pyrazolyl, oxazolyl, thiazolyl, pyridinyl, pyrimidinyl, tetrahydropyrimidinyl, pyrazinyl, dihydroisooxazolyl, isooxazolyl, isothiazolyl, imidazolyl, oxadiazolyl, thiadiazolyl, imidazolinyl, Imidazolidinyl, oxazolinyl, pyrazolinyl, thiazolinyl, triazolinyl, dihydrobenzomxazolyl, dihydrobenzoisoxazole, dihydrobenzothiazolyl, dihydrooxazolopyridinyl, dihydroimidazopyridinyl, dihydropyrazolopyridinyl, dihydroindazolyl, dihydrobenzoisothiazolyl, dihydroisothiazolopyridine, indazolyl, benzotriazolyl, or triazolopyridine, each of which is substituted by one, two, three, or four substituents independently selected from R².

In certain embodiments, A¹ is

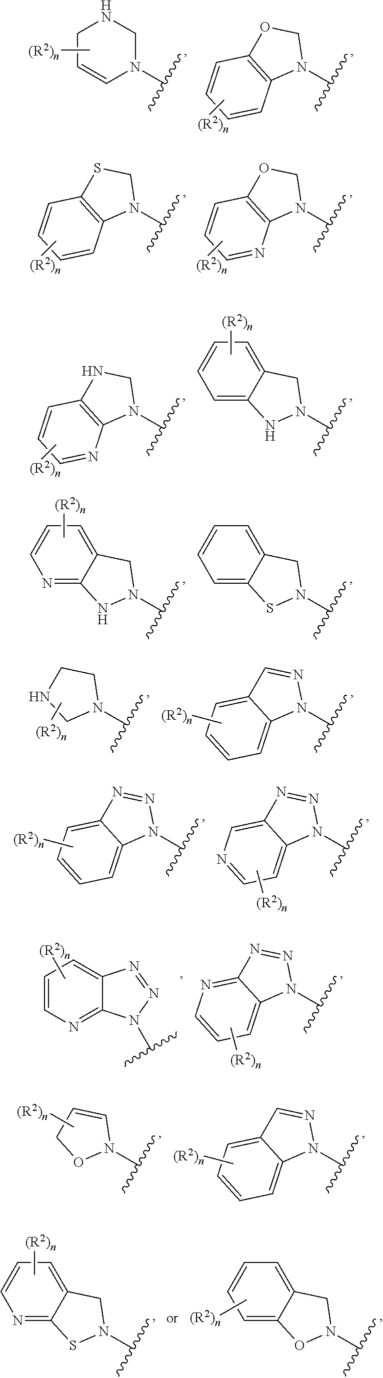

wherein n is 0, 1, 2, 3, or 4.

In certain embodiments, the acid ceramidase inhibitor is a compound of Formula I-1:

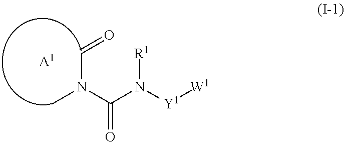

or a pharmaceutically acceptable salt thereof wherein:

A¹ is a cyclic group selected from 5-6 membered heterocyclyl, 5-6 membered heteroaryl, and bicyclic heterocyclyl, each of which is substituted by 1, 2, or 3 occurrences of R²;

R¹ represents independently for each occurrence hydrogen, C₁₋₄alkyl, —C₁₋₄alkyl-phenyl, —CO₂—C₁₋₆alkyl, —C(O)—NH₂, —C(O)—NH—C₁₋₆alkyl, or —C(O)—N(C₁₋₆alkyl)₂;

R² represents independently for each occurrence R¹, C₁₋₄alkyl, C₁₋₄haloalkyl, C₁₋₄alkoxy, halogen, hydroxyl, oxo, cyano, nitro, azido, —N(R¹)₂, —C(O)—C₁₋₄alkyl, —C(O)-phenyl, —CO₂—R¹, —C(O)—NH₂, —C(O)—NH—C₁₋₆alkyl, —C(O)—N(C₁₋₆alkyl)₂, —O—C(O)—NH₂, —O—C(O)—NH—C₁₋₆alkyl, —O—C(O)—N(C₁₋₆alkyl)₂, —C₁₋₄alkyl-phenyl, C₃₋₁₀cycloalkyl, C₃₋₁₀heterocyclyl, 6-10 membered aryl, 6-10 membered heteroaryl, —C₁₋₄alkylene-C₃₋₁₀cycloalkyl, —C₁₋₄alkylene-C₃₋₁₀heterocyclyl, —C₁₋₄alkylene-6-10 membered aryl, or —C₁₋₄alkylene-6-10 membered heteroaryl;

Y¹ represents:
C₁₋₁₈alkylene, C₂₋₁₈alkenylene, or C₂₋₁₈alkynylene;
C₃₋₁₀cycloalkylene, 3-10 membered, 6-10 membered arylene, or 6-10 membered heteroarylene, each of which is substituted by 0, 1, 2, or 3 occurrences of C₁₋₄alkyl; or
R¹ and Y¹ together with the nitrogen to which they are attached form a 3-10 membered heterocyclylene; and

W¹ represents:
hydrogen; or
C₃₋₁₀cycloalkylene, C₃₋₁₀heterocyclylene, 6-10 membered arylene, or 6-10 membered heteroarylene.

Definitions of the variables in Formula I-1above encompass multiple chemical groups. The application contemplates embodiments where, for example, i) the definition of a variable is a single chemical group selected from those chemical groups set forth above, ii) the definition is a collection of two or more of the chemical groups selected from those set forth above, and iii) the compound is defined by a combination of variables in which the variables are defined by (i) or (ii), e.g., such as where Y¹ is C₁₋₁₈alkylene, W¹ is 6-10 membered arylene, and A¹ is bicyclic heterocyclyl.

In certain embodiments, A¹ is

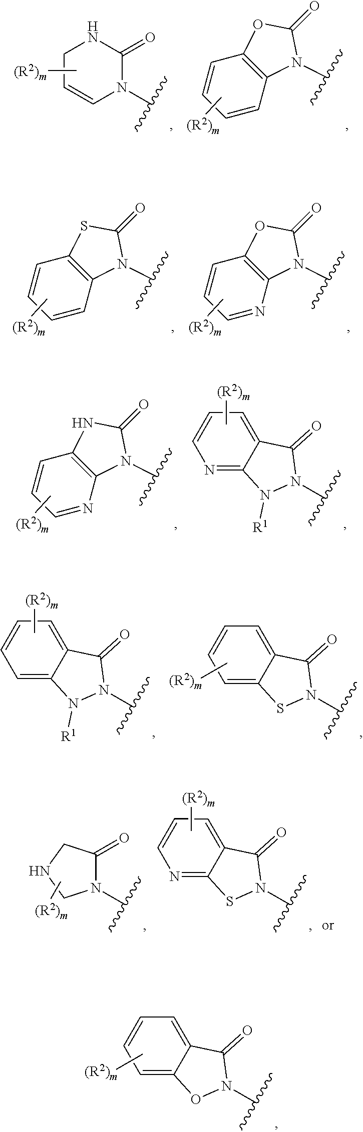

wherein m is 0, 1, 2, 3.

In certain embodiments, an acid ceramidase inhibitor may be selected from the group consisting of:

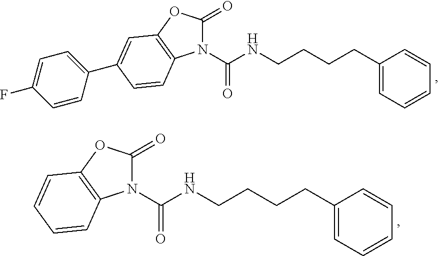 [SMILES: O=C(NCCCCc1ccccc1)n1c(=O)oc2cc(-c3ccc(F)cc3)ccc21 ; O=C(NCCCCc1ccccc1)n1c(=O)oc2ccccc21]

and pharmaceutically acceptable salts thereof.

In certain embodiments, an acid ceramidase inhibitor may be selected from the group consisting of:

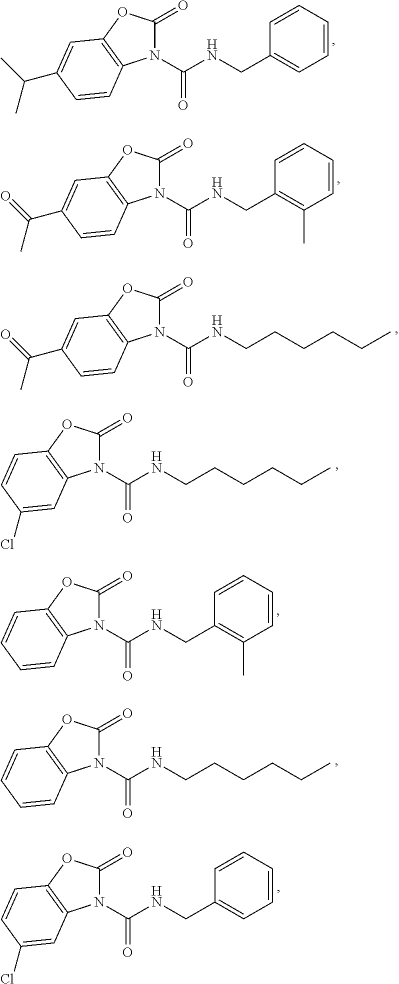

and pharmaceutically acceptable salts thereof.

In certain embodiments, an acid ceramidase inhibitor may be selected from the group consisting of:

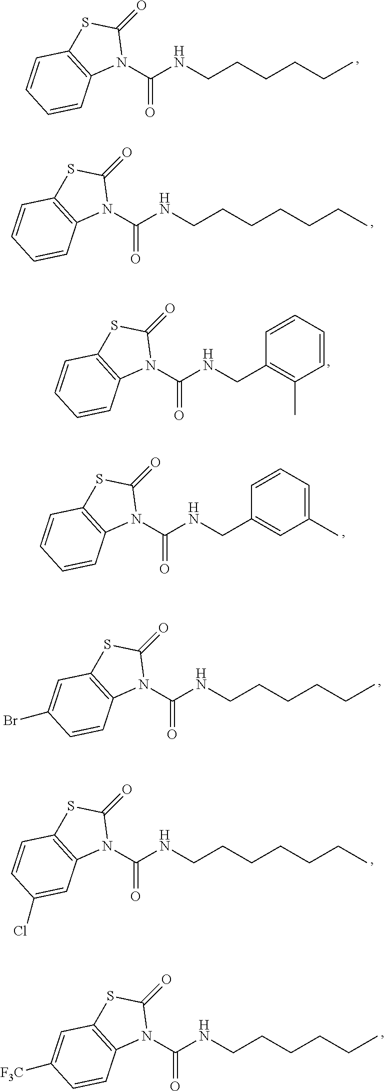

and pharmaceutically acceptable salts thereof.

In certain embodiments, an acid ceramidase inhibitor may be selected from the group consisting of:

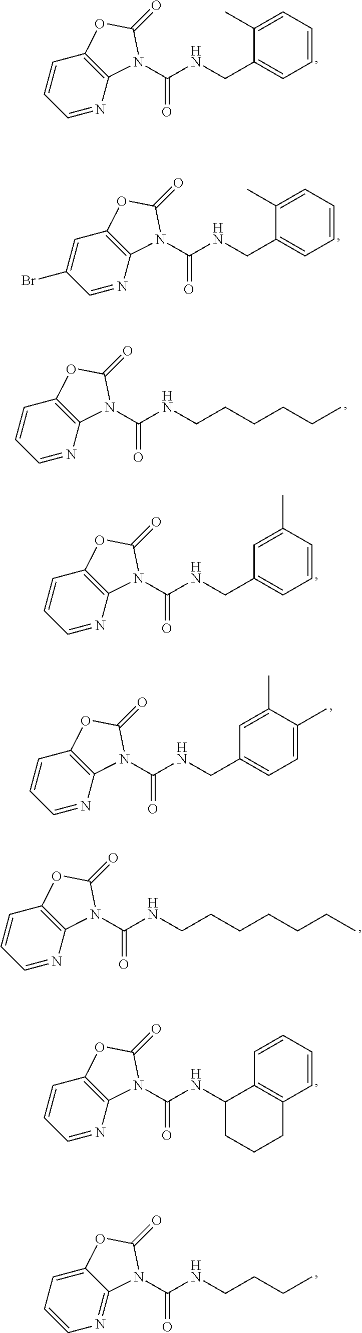

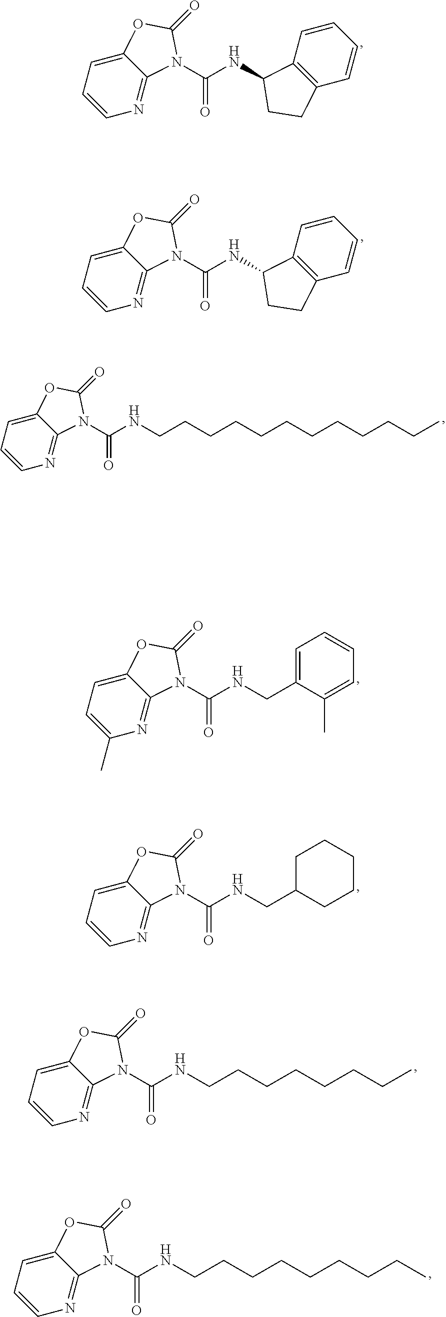

and pharmaceutically acceptable salts thereof.

In certain embodiments, an acid ceramidase inhibitor may be selected from the group consisting of:

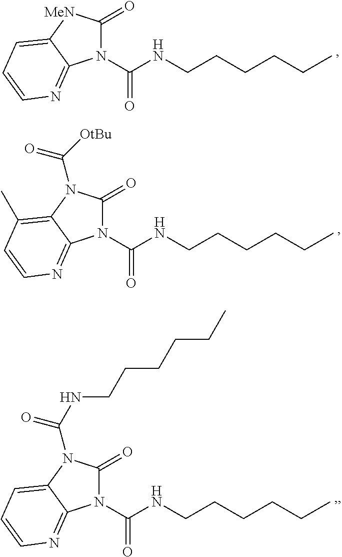

and pharmaceutically acceptable salts thereof.

In certain embodiments, an acid ceramidase inhibitor may be selected from the group consisting of:

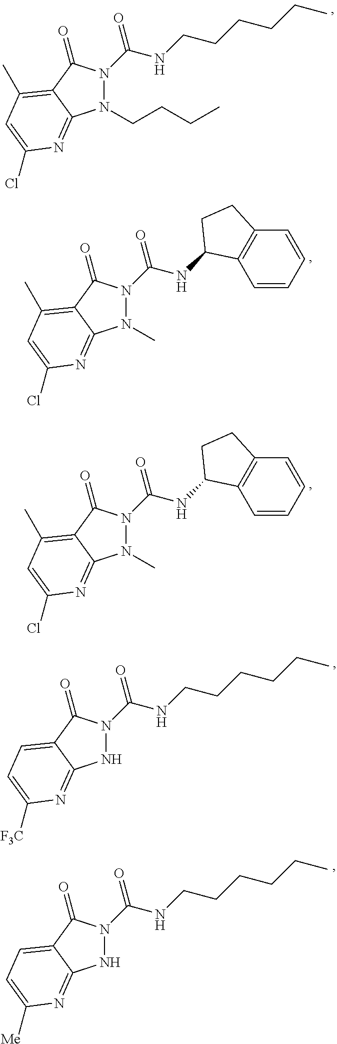

and pharmaceutically acceptable salts thereof.

In certain embodiments, an acid ceramidase inhibitor may be selected from the group consisting of:

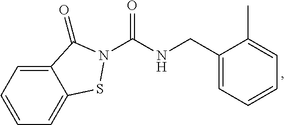 [SMILES: Cc1ccccc1CNC(=O)n1sc2ccccc2c1=O]

and pharmaceutically acceptable salts thereof.

In certain embodiments, an acid ceramidase inhibitor may be selected from the group consisting of:

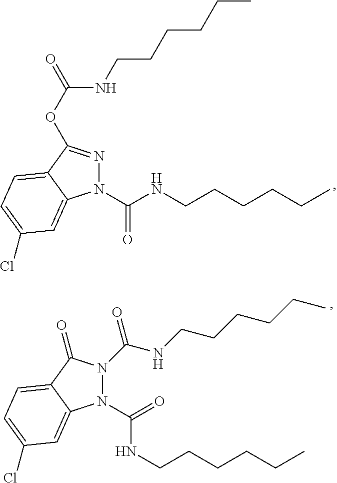 [SMILES: CCCCCCNC(=O)Oc1nn(C(=O)NCCCCCC)c2cc(Cl)ccc12 ; CCCCCCNC(=O)n1c(=O)c2ccc(Cl)cc2n1C(=O)NCCCCCC]

and pharmaceutically acceptable salts thereof.

In certain embodiments, an acid ceramidase inhibitor may be selected from the group consisting of:

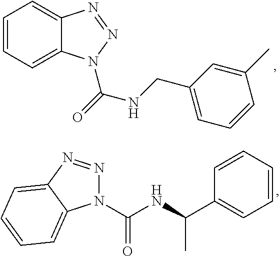

and pharmaceutically acceptable salts thereof.

In certain embodiments, an acid ceramidase inhibitor may be selected from the group consisting of:

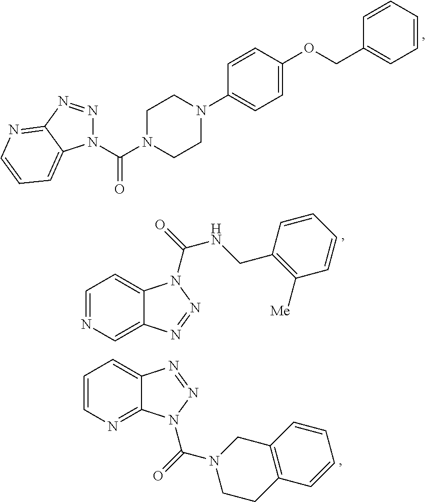

and pharmaceutically acceptable salts thereof.

In certain embodiments, an acid ceramidase inhibitor may be selected from the group consisting of:

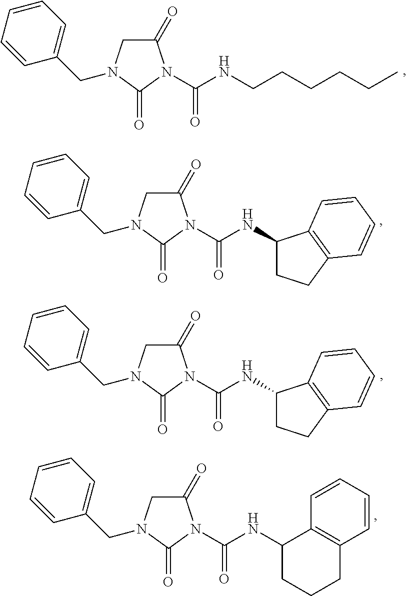

and pharmaceutically acceptable salts thereof.

In certain embodiments, an acid ceramidase inhibitor may be selected from the group consisting of:

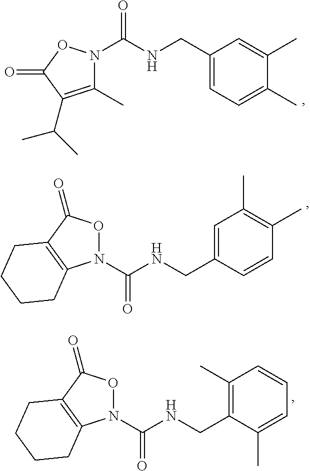

and pharmaceutically acceptable salts thereof.

In certain embodiments, an acid ceramidase inhibitor may be selected from the group consisting of:

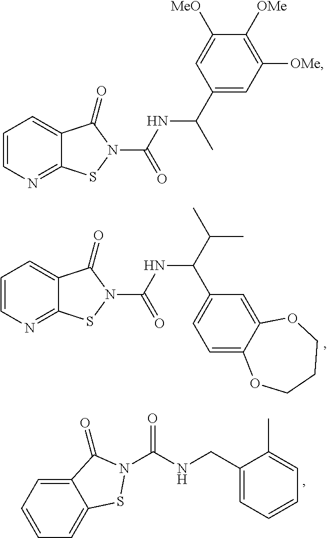

and pharmaceutically acceptable salts thereof.

In certain embodiments, an acid ceramidase inhibitor may be selected from the group consisting of:

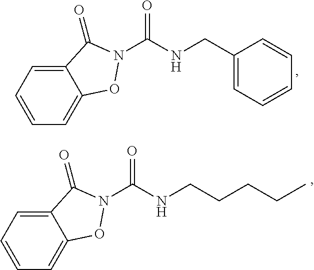

and pharmaceutically acceptable salts thereof.

In certain embodiments, an acid ceramidase inhibitor may be selected from the group consisting of:

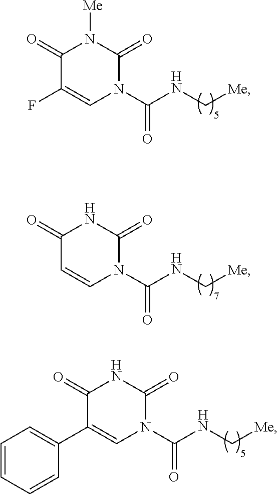

and pharmaceutically acceptable salts thereof.

Other contemplated acid ceramidase inhibitors may be selected from the group consisting of:

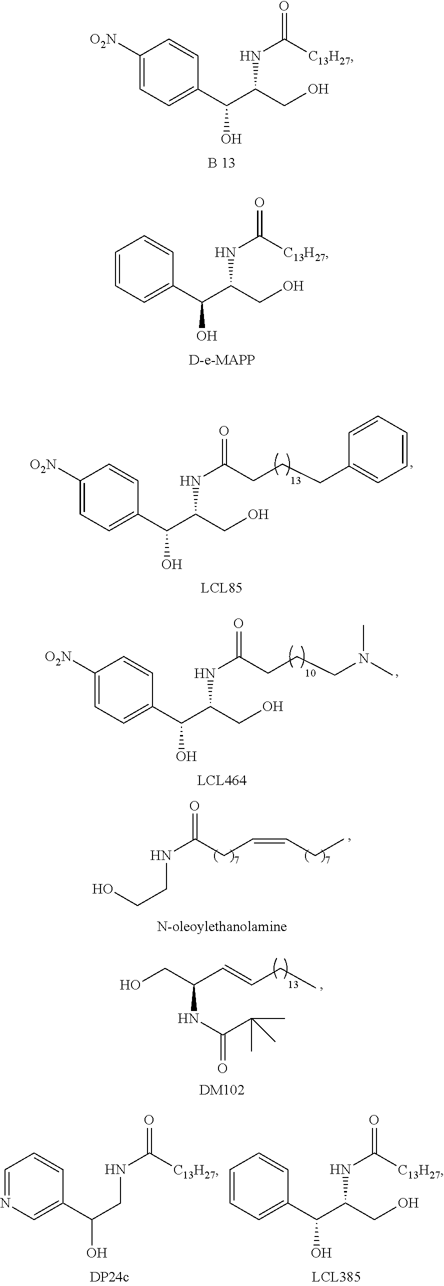

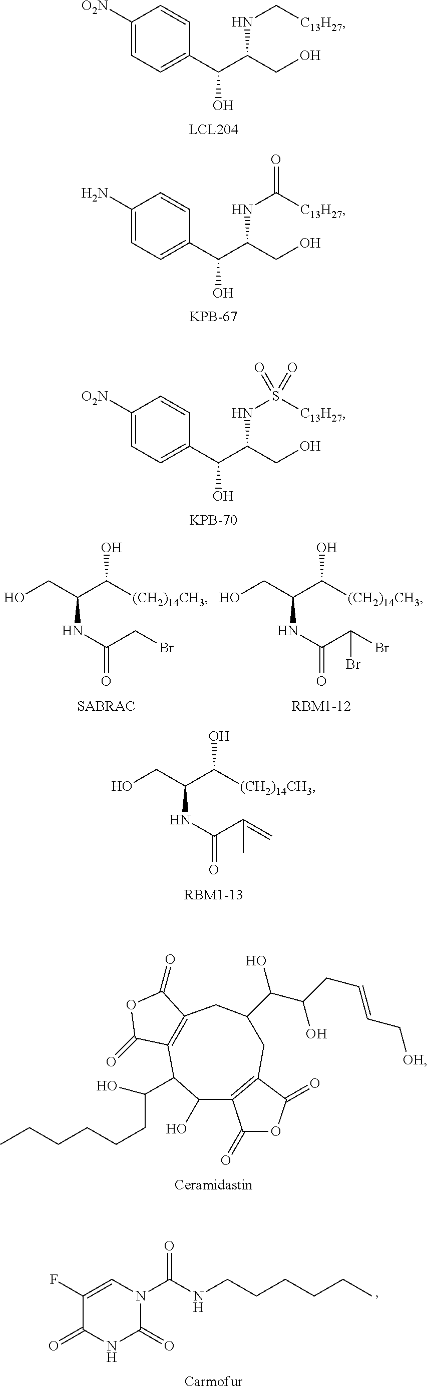

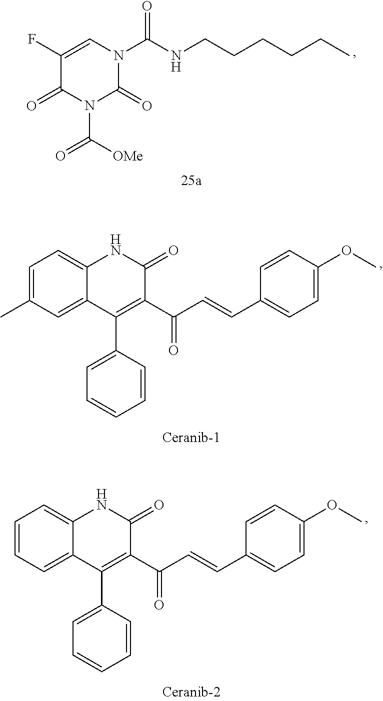

and pharmaceutically acceptable salts thereof.

When the acid ceramidase inhibitor is delivered by systemic administration, the inhibitor preferably is capable of traversing the blood brain barrier.

In one embodiment, an exemplary acid ceramidase inhibitor is a compound of Formula III:

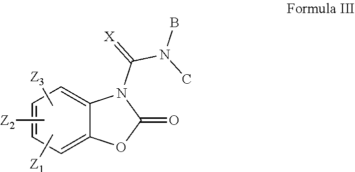

or a pharmaceutically acceptable salt thereof, wherein:

X is O or S;

B is hydrogen, linear or branched C₁₋₆ alkyl;

C is a linear or branched C₅₋₁₂ alkyl group or a group:

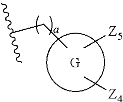

wherein:

a is an integer from 1 to 6;

G is a 3-10 membered saturated, unsaturated, aromatic or heteroaromatic, single or fused ring comprising up to three heteroatoms selected from N, O, S; and Z₄ and Z₅ are as defined below;

Z₁, Z₂, Z₃, Z₄, and Z₅, are independently selected from the group consisting of hydrogen, halogen, linear or branched C₁₋₆ alkyl, optionally substituted cycloalkyl C₁₋₆ alkyl, optionally substituted cycloalkyl C₂₋₆ alkenyl, optionally substituted aryl C₁₋₆ alkyl, optionally substituted aryl C₁₋₆ alkenyl, C₁₋₆ alkoxy, optionally substituted cycloalkyl C₁₋₆ alkoxy, optionally substituted aryl C₁₋₆ alkoxy, hydroxy C₁₋₆ alkyl, OH, CN, NO, fluoro C₁₋₆ alkyl, fluoro C₁₋₆ alkoxy, optionally substituted aryl, C₁₋₆ alkylCO, optionally substituted arylCO, optionally substituted aryl C₁₋₆₄ alkylCO, COOZ₇, CONZ₈Z₉, SO₂Z₁₀;

wherein Z₇, Z₈, Z₉ and Z₁₀ are independently selected from the group consisting of hydrogen, linear or branched C₁₋₆ alkyl;

Z₁, Z₂, Z₃, Z₄ and Z₅ can be attached to any position of the ring to which they are connected.

In certain embodiments, compounds of Formula (III) as defined above are provided wherein:

X is O or S;

B is hydrogen or a linear or branched C₁₋₆ alkyl;

C is a linear or branched C₅₋₁₂ alkyl group or a group:

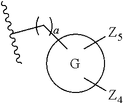

wherein:

a is an integer from 1 to 6;

G is
(i) an optionally substituted C₃-C₁₀ cycloalkyl which is cyclopropane, cyclobutane, cyclopentane, cyclopentene, cyclohexane, cyclohexene, cyclohexadiene, or cycloheptane;
(ii) an optionally substituted aryl which is phenyl, alpha- or beta-naphthyl, 9,10-dihydroanthracenyl, indanyl, fluorenyl or biphenyl; an optionally substituted heteroaryl which is pyrrolyl, furyl, thiophenyl, imidazolyl, pyrazolyl, oxazolyl, isoxazolyl, thiazolyl, isothiazolyl, indolyl, benzofuranyl, benzothiophenyl, benzimidazolyl, benzopyrazolyl, benzoxazolyl, benzoisoxazolyl, benzothiazolyl, benzoisothiazolyl, triazolyl, oxadiazolyl, tetrazolyl, thienyl, pyridyl, pyrazinyl, pyrimidinyl, pyridazinyl, quinolinyl, isoquinolinyl, quinazolinyl or quinoxalinyl; or
(iii) an optionally substituted heterocyclic ring which is oxirane, aziridine, oxetane, azetidine, tetrahydrofuran, dihydrofuran, tetrahydrothiophene, dihydrothiophene, pyrrolidine, dihydropyrrole, pyran, dihydropyran, tetrahydropyran, tetrahydrothiopyran, piperidine, pyrazoline, oxazoline, isoxazolidine, isoxazoline, thiazolidine, thiazoline, isothiazoline, dioxane, piperazine, morpholine, thiomorpholine, hexamethyleneimine or homopiperazine;

Z₁, Z₂, Z₃, Z₄ and Z₅, are independently selected from the group consisting of hydrogen, halogen, linear or branched C₁₋₆ alkyl, optionally substituted cycloalkyl C₁₋₆ alkyl, optionally substituted cycloalkyl C₂₋₆ alkenyl, optionally substituted aryl C₁₋₆ alkyl, optionally substituted aryl C₂₋₆ alkenyl, C₁₋₆ alkoxy, optionally substituted cycloalkyl C₁₋₆ alkoxy, optionally substituted aryl C₁₋₆ alkoxy, hydroxy C₁₋₆ alkyl, OH, CN, NO₂, fluoro C₁₋₆ alkyl, fluoro C₁₋₆ alkoxy, optionally substituted aryl, C₁₋₆ alkylCO, optionally substituted arylCO, optionally substituted aryl C₁₋₆ alkylCO, COOZ₇, CONZ₈Z₉, SO₂Z₁₀;

wherein Z₇, Z₈, Z₉ and Z₁₀ are independently selected from the group consisting of hydrogen, linear or branched C₁₋₆ alkyl;

wherein Z₁, Z₂, Z₃, Z₄ and Z₅ can be attached to any position of the ring to which they are connected.

In certain embodiments, compounds of Formula (III) as defined above are provided wherein:

X is O or S;

B is hydrogen or a linear or branched C₁₋₆ alkyl;

C is a linear or branched C₅₋₉ alkyl group or a group:

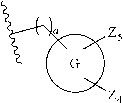

wherein:

a is an integer from 1 to 6;

G is an aryl selected from naphthyl or phenyl, (C₃-C₁₀)cycloalkyl, a heteroaryl which is pyridyl, thiophenyl, pyrimidinyl, furyl, indolyl;

wherein Z₄ and Z₅, if present, independently are halogen, NO₂, (C₁-C₃)alkoxy-, (C₃-C₁₀) cycloalkyl, linear or branched C₁-C₆ alkyl;

Z₄ and Z₅ can be attached to any position of the ring to which they are connected;

Z₁, Z₂, Z₃, are independently (i) hydrogen, halogen, linear or branched C₁₋₆ alkyl, OH, CN, NO₂, fluoro C₁₋₆ alkyl, hydroxy C₁₋₆ alkyl; (ii) phenyl optionally substituted with C₁-C₆ alkyl, C₁-C₃ alkoxy, C₂-C₆ alkenyl, halogen, NO₂, CF₃; (ii) phenyl C₁₋₆ alkyl optionally substituted with C₁-C₆ alkyl, C₁-C₃ alkoxy, C₂-C₆ alkenyl, halogen, NO₂, CF₃; (iv) phenyl C₂₋₆ alkenyl optionally substituted with C₁-C₆ alkyl, C₁-C₃ alkoxy, C₂-C₆ alkenyl, halogen, NO₂, CF₃; (v) phenyl CO optionally substituted with C₁-C₆ alkyl, C₁-C₃ alkoxy, C₂-C₆ alkenyl, halogen, NO₂, CF₃; (vi) C₁-C₆ alkyl CO optionally substituted with phenyl, optionally substituted with C₁-C₆ alkyl, C₁-C₃ alkoxy, C₂-C₆ alkenyl, halogen, NO₂, CF₃; (vii) (C₃-C₁₀)cycloalkyl C₁₋₆ alkyl optionally substituted with C₁-C₆ alkyl, C₂-C₆ alkenyl, halogen; (viii) (C₃-C₁₀)cycloalkyl C₂₋₆ alkenyl optionally substituted with C₁-C₆ alkyl, C₂-C₆ alkenyl, halogen; or (iX) C₁₋₆ alkoxy optionally substituted with halogen, (C₃-C₁₀)cycloalkyl, phenyl;

wherein Z₁, Z₂, or Z₃ can be attached to any position of the ring to which they are connected.

In certain embodiments, compounds of Formula (III) as defined above are provided

wherein:

X is O;

B is hydrogen;

C is a linear or branched C₅₋₉ alkyl group or preferably a group:

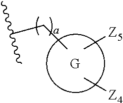

wherein:

a is an integer from 1 to 4;

G is phenyl, thiophenyl, pyridyl, naphthyl or C₃₋₇ cycloalkyl, preferably cyclohexyl;

Z₁, Z₂, Z₃, Z₄ and Z₅, are, independently, H, F, Cl, Br, Me, Et, Pr, MeO, BuO, OH, CN, NO₂, CF₃, Ph, MeCO, or EtCO;

wherein Z₁, Z₂, Z₃, Z₄ and Z₅ can be attached to any position of the ring to which they are connected.

Exemplary compounds of Formula III are set forth in Table 1.

TABLE 1
Exemplary compounds of Formula III

| Example | Structure | Formula | MW | Name |
|---|---|---|---|---|
| 1 | 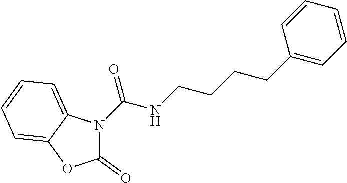 [SMILES: O=C(NCCCCc1ccccc1)n1c(=O)oc2ccccc21] | C₁₈H₁₈N₂O₃ | 310.4 | 2-oxo-N-(4-phenylbutyl)-1,3- benzoxazole-3-carboxamide |
| 2 | 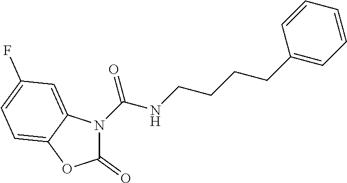 [SMILES: O=C(NCCCCc1ccccc1)n1c(=O)oc2ccc(F)cc21] | C₁₈H₁₇FN₂O₃ | 328.3 | 5-fluoro-2-oxo-N-(4- phenylbutyl)-1,3-benzoxazole- 3-carboxamide |
| 3 | 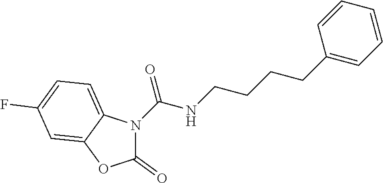 [SMILES: O=C(NCCCCc1ccccc1)n1c(=O)oc2cc(F)ccc21] | C₁₈H₁₇FN₂O₃ | 328.3 | 6-fluoro-2-oxo-N-(4- phenylbutyl)-1,3-benzoxazole- 3-carboxamide |
| 4 | 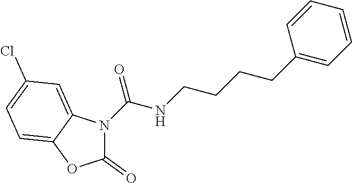 [SMILES: O=C(NCCCCc1ccccc1)n1c(=O)oc2ccc(Cl)cc21] | C₁₈H₁₇ClN₂O₃ | 344.8 | 5-chloro-2-oxo-N-(4- phenylbutyl)-1,3-benzoxazole- 3-carboxamide |
| 5 | 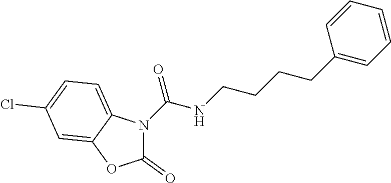 [SMILES: O=C(NCCCCc1ccccc1)n1c(=O)oc2cc(Cl)ccc21] | C₁₈H₁₇ClN₂O₃ | 344.8 | 6-chloro-2-oxo-N-(4- phenylbutyl)-1,3-benzoxazole- 3-carboxamide |
| 6 | 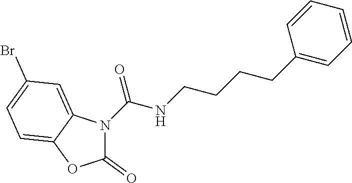 [SMILES: O=C(NCCCCc1ccccc1)n1c(=O)oc2ccc(Br)cc21] | C₁₈H₁₇BrN₂O₃ | 389.2 | 5-bromo-2-oxo-N-(4- phenylbutyl)-1,3-benzoxazole- 3-carboxamide |
| 7 | 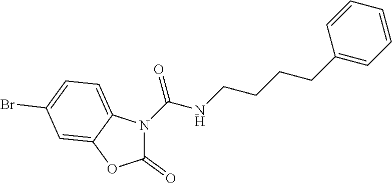 [SMILES: O=C(NCCCCc1ccccc1)n1c(=O)oc2cc(Br)ccc21] | C₁₈H₁₇BrN₂O₃ | 389.2 | 6-bromo-2-oxo-N-(4- phenylbutyl)-1,3-benzoxazole- 3-carboxamide |
| 8 | 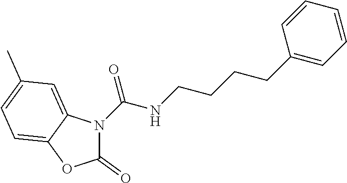 [SMILES: Cc1ccc2oc(=O)n(C(=O)NCCCCc3ccccc3)c2c1] | C₁₉H₂₀N₂O₃ | 324.4 | 5-methyl-2-oxo-N-(4- phenylbutyl)-1,3-benzoxazole- 3-carboxamide |
| 9 | 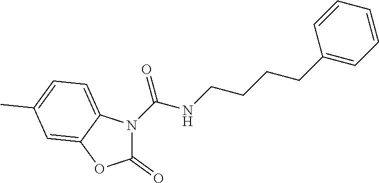 [SMILES: Cc1ccc2c(c1)oc(=O)n2C(=O)NCCCCc1ccccc1] | C₁₉H₂₀N₂O₃ | 324.4 | 6-methyl-2-oxo-N-(4- phenylbutyl)-1,3-benzoxazole- 3-carboxamide |
| 10 | 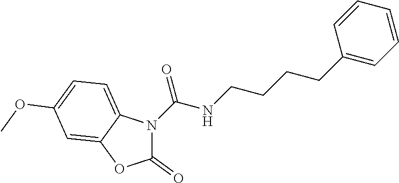 [SMILES: COc1ccc2c(c1)oc(=O)n2C(=O)NCCCCc1ccccc1] | C₁₉H₂₀N₂O₄ | 340.4 | 6-methoxy-2-oxo-N-(4- phenylbutyl)-1,3-benzoxazole- 3-carboxamide |
| 11 | 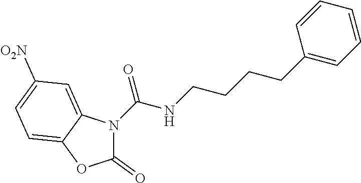 [SMILES: O=C(NCCCCc1ccccc1)n1c(=O)oc2ccc([N+](=O)[O-])cc21] | C₁₈H₁₇N₃O₅ | 355.3 | 5-nitro-2-oxo-N-(4- phenylbutyl)-1,3-benzoxazole- 3-carboxamide |
| 12 | 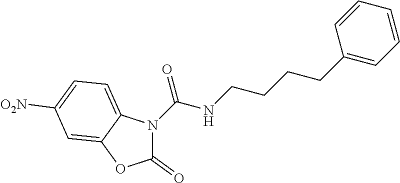 [SMILES: O=C(NCCCCc1ccccc1)n1c(=O)oc2cc([N+](=O)[O-])ccc21] | C₁₈H₁₇N₃O₅ | 355.3 | 6-nitro-2-oxo-N-(4- phenylbutyl)-1,3-benzoxazole- 3-carboxamide |
| 13 | 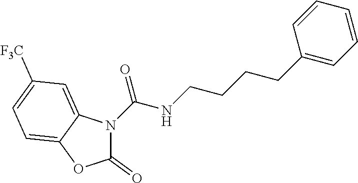 [SMILES: O=C(NCCCCc1ccccc1)n1c(=O)oc2ccc(C(F)(F)F)cc21] | C₁₉H₁₇F₃N₂O₃ | 378.3 | 2-oxo-N-(4-phenylbutyl)-5- (trifluoromethyl)-1,3- benzoxazole-3-carboxamide |
| 14 | 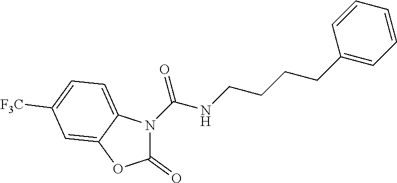 [SMILES: O=C(NCCCCc1ccccc1)n1c(=O)oc2cc(C(F)(F)F)ccc21] | C₁₉H₁₇F₃N₂O₃ | 378.3 | 2-oxo-N-(4-phenylbutyl)-6- (trifluoromethyl)-1,3- benzoxazole-3-carboxamide |
| 15 | 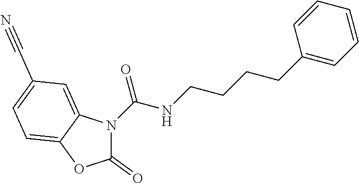 [SMILES: N#Cc1ccc2oc(=O)n(C(=O)NCCCCc3ccccc3)c2c1] | C₁₉H₁₇N₃O₃ | 335.4 | 6-cyano-2-oxo-N-(4- phenylbutyl)-1,3-benzoxazole- 3-carboxamide |
| 16 | 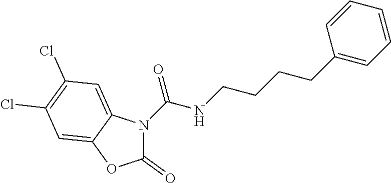 [SMILES: O=C(NCCCCc1ccccc1)n1c(=O)oc2cc(Cl)c(Cl)cc21] | C₁₈H₁₆Cl₂N₂O₃ | 379.2 | 5,6-dichloro-2-oxo-N-(4- phenylbutyl)-1,3-benzoxazole- 3-carboxamide |
| 17 | 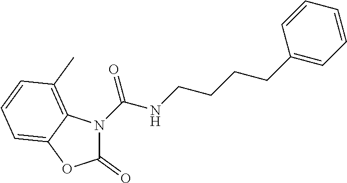 [SMILES: Cc1cccc2oc(=O)n(C(=O)NCCCCc3ccccc3)c12] | C₁₉H₂₀N₂O₃ | 324.4 | 4-methyl-2-oxo-N-(4- phenylbutyl)-1,3-benzoxazole- 3-carboxamide |
| 18 | 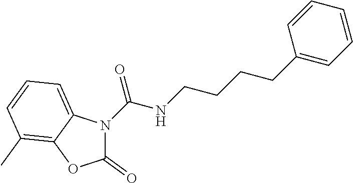 [SMILES: Cc1cccc2c1oc(=O)n2C(=O)NCCCCc1ccccc1] | C₁₉H₂₀N₂O₃ | 324.4 | 7-methyl-2-oxo-N-(4- phenylbutyl)-1,3-benzoxazole- 3-carboxamide |
| 19 | 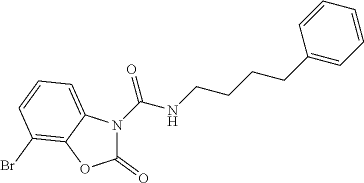 [SMILES: O=C(NCCCCc1ccccc1)n1c(=O)oc2c(Br)cccc21] | C₁₈H₁₇BrN₂O₃ | 389.2 | 7-bromo-2-oxo-N-(4- phenylbutyl)-1,3-benzoxazole- 3-carboxamide |
| 20 | 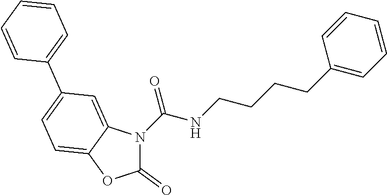 [SMILES: O=C(NCCCCc1ccccc1)n1c(=O)oc2ccc(-c3ccccc3)cc21] | C₂₄H₂₂N₂O₃ | 386.4 | 2-oxo-5-phenyl-N-(4- phenylbutyl)-1,3-benzoxazole- 3-carboxamide |
| 21 | 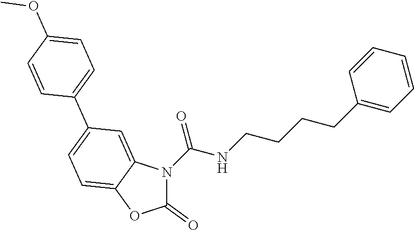 [SMILES: COc1ccc(-c2ccc3oc(=O)n(C(=O)NCCCCc4ccccc4)c3c2)cc1] | C₂₅H₂₄N₂O₄ | 416.5 | 5-(4-methoxyphenyl)-2-oxo-N- (4-phenylbutyl)-1,3- benzoxazole-3-carboxamide |
| 22 | 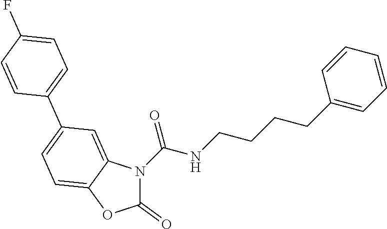 [SMILES: O=C(NCCCCc1ccccc1)n1c(=O)oc2ccc(-c3ccc(F)cc3)cc21] | C₂₄H₂₁FN₂O₃ | 404.4 | 5-(4-fluorophenyl)-2-oxo-N- (4-phenylbutyl)-1,3- benzoxazole-3-carboxamide |
| 23 | 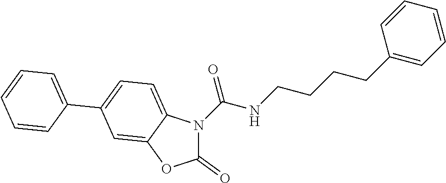 [SMILES: O=C(NCCCCc1ccccc1)n1c(=O)oc2cc(-c3ccccc3)ccc21] | C₂₄H₂₂N₂O₃ | 386.4 | 2-oxo-6-phenyl-N-(4- phenylbutyl)-1,3-benzoxazole- 3-carboxamide |
| 24 | 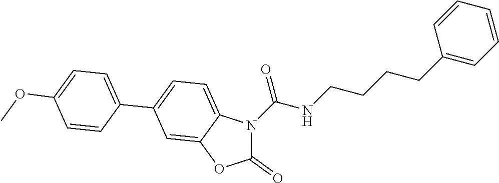 [SMILES: COc1ccc(-c2ccc3c(c2)oc(=O)n3C(=O)NCCCCc2ccccc2)cc1] | C₂₅H₂₄N₂O₄ | 416.5 | 6-(4-methoxyphenyl)-2-oxo-N- (4-phenylbutyl)-1,3- benzoxazole-3-carboxamide |
| 25 | 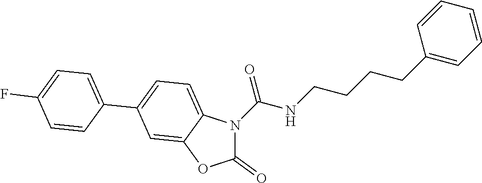 [SMILES: O=C(NCCCCc1ccccc1)n1c(=O)oc2cc(-c3ccc(F)cc3)ccc21] | C₂₄H₂₁FN₂O₃ | 404.4 | 6-(4-fluorophenyl)-2-oxo-N- (4-phenylbutyl)-1,3- benzoxazole-3-carboxamide |
| 26 | 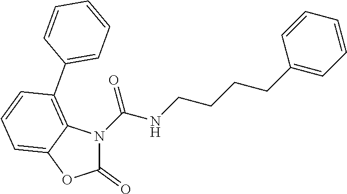 [SMILES: O=C(NCCCCc1ccccc1)n1c(=O)oc2cccc(-c3ccccc3)c21] | C₂₄H₂₂N₂O₃ | 386.4 | 2-oxo-4-phenyl-N-(4- phenylbutyl)-1,3-benzoxazole- 3-carboxamide |
| 27 | 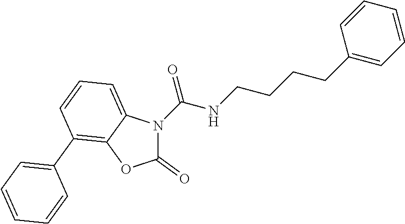 [SMILES: O=C(NCCCCc1ccccc1)n1c(=O)oc2c(-c3ccccc3)cccc21] | C₂₄H₂₂N₂O₃ | 386.4 | 2-oxo-7-phenyl-N-(4- phenylbutyl)-1,3-benzoxazole- 3-carboxamide |
| 28 | 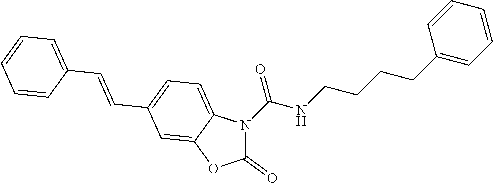 [SMILES: O=C(NCCCCc1ccccc1)n1c(=O)oc2cc(/C=C/c3ccccc3)ccc21] | C₂₆H₂₄N₂O₃ | 412.5 | 2-oxo-N-(4-phenylbutyl)-6- [(E)-styryl]-1,3-benzoxazole- 3-carboxamide |
| 29 | 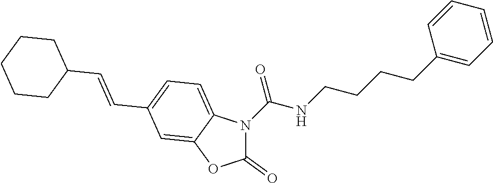 [SMILES: O=C(NCCCCc1ccccc1)n1c(=O)oc2cc(/C=C/C3CCCCC3)ccc21] | C₂₆H₃₀N₂O₃ | 418.5 | 6-[(E)-2-cyclohexylvinyl]-2- oxo-N-(4-phenylbutyl)-1,3- benzoxazole-3-carboxamide |
| 30 | 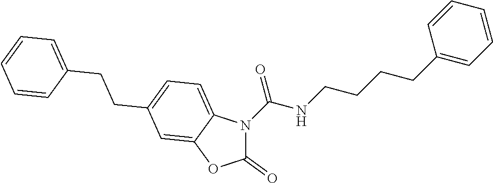 [SMILES: O=C(NCCCCc1ccccc1)n1c(=O)oc2cc(CCc3ccccc3)ccc21] | C₂₆H₂₆N₂O₃ | 414.5 | 2-oxo-6-phenethyl-N-(4- phenylbutyl)-1,3-benzoxazole- 3-carboxamide |
| 31 | 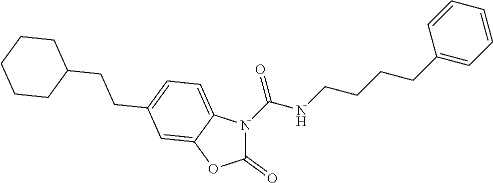 [SMILES: O=C(NCCCCc1ccccc1)n1c(=O)oc2cc(CCC3CCCCC3)ccc21] | C₂₆H₃₂N₂O₃ | 420.5 | 6-(2-cyclohexylthyl)-2-oxo-N- (4-phenylbutyl)-1,3- benzoxazole-3-carboxamide |
| 32 | 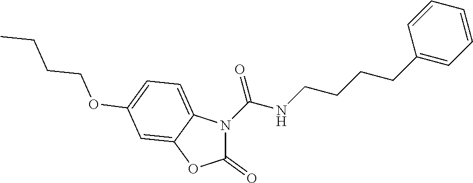 [SMILES: CCCCOc1ccc2c(c1)oc(=O)n2C(=O)NCCCCc1ccccc1] | C₂₂H₂₆N₂O₄ | 382.4 | 6-butoxy-2-oxo-N-(4- phenylbutyl)-1,3-benzoxazole- 3-carboxamide |
| 33 | 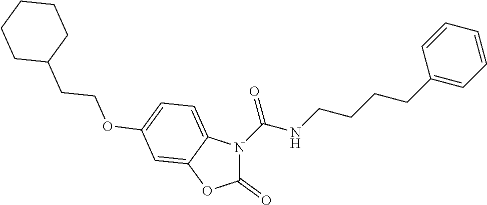 [SMILES: O=C(NCCCCc1ccccc1)n1c(=O)oc2cc(OCCC3CCCCC3)ccc21] | C₂₆H₃₂N₂O₄ | 436.5 | 6-(2-cyclohexylethoxy)-2-oxo- N-(4-phenylbutyl)-1,3- benzoxazole-3-carboxamide |
| 34 | 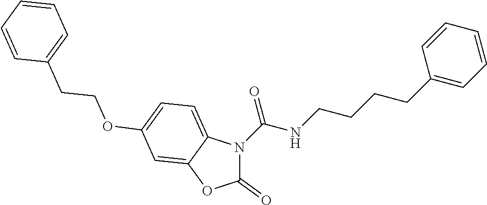 [SMILES: O=C(NCCCCc1ccccc1)n1c(=O)oc2cc(OCCc3ccccc3)ccc21] | C₂₆H₂₆N₂O₄ | 430.5 | 2-oxo-6-phenethyloxy-N-(4- phenylbutyl)-1,3-benzoxazole- 3-carboxamide |
| 35 | 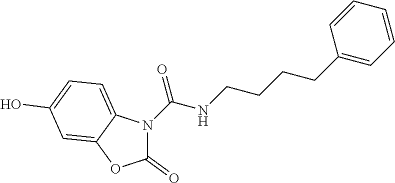 [SMILES: O=C(NCCCCc1ccccc1)n1c(=O)oc2cc(O)ccc21] | C₁₈H₁₈N₂O₄ | 326.4 | 6-hydroxy-2-oxo-N-(4- phenylbutyl)-1,3-benzoxazole- 3-carboxamide |
| 36 | 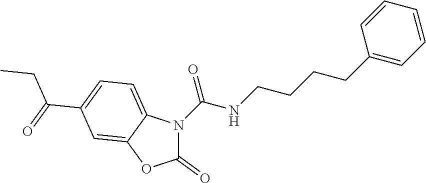 [SMILES: CCC(=O)c1ccc2c(c1)oc(=O)n2C(=O)NCCCCc1ccccc1] | C₂₁H₂₂N₂O₄ | 366.4 | 2-oxo-N-(4-phenylbutyl)-6- propanoyl-1,3-benzoxazole-3- carboxamide |
| 37 | 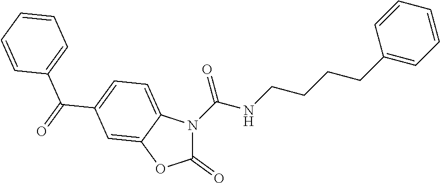 [SMILES: O=C(c1ccccc1)c1ccc2c(c1)oc(=O)n2C(=O)NCCCCc1ccccc1] | C₂₅H₂₂N₂O₄ | 414.4 | 6-benzoyl-2-oxo-N-(4- phenylbutyl)-1,3-benzoxazole- 3-carboxamide |
| 38 | 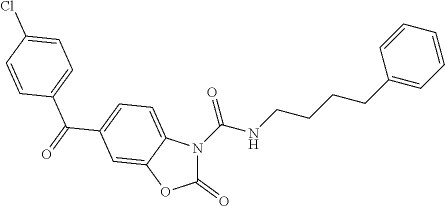 [SMILES: O=C(c1ccc(Cl)cc1)c1ccc2c(c1)oc(=O)n2C(=O)NCCCCc1ccccc1] | C₂₅H₂₁ClN₂O₄ | 448.9 | 6-(4-chlorobenzoyl)-2-oxo-N- (4-phenylbutyl)-1,3- benzoxazole-3-carboxamide |
| 39 | 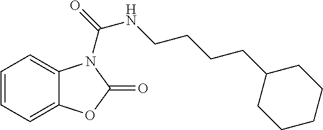 [SMILES: O=C(NCCCCC1CCCCC1)n1c(=O)oc2ccccc21] | C₁₈H₂₄N₂O₃ | 316.4 | N-(4-cyclohexylbutyl)-2-oxo- 1,3-benzoxazole-3- carboxamide |
| 40 | 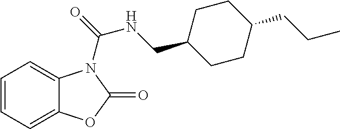 [SMILES: CCC[C@H]1CC[C@H](CNC(=O)n2c(=O)oc3ccccc32)CC1] | C₁₈H₂₄N₂O₃ | 316.4 | 2-oxo-N-[(4- propylcyclohexyl)methyl]-1,3- benzoxazole-3-carboxamide |
| 41 | 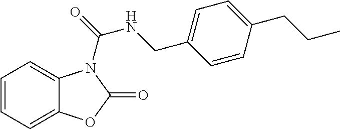 [SMILES: CCCc1ccc(CNC(=O)n2c(=O)oc3ccccc32)cc1] | C₁₈H₁₈N₂O₃ | 310.4 | 2-oxo-N-[(4- propylphenyl)methyl]-1,3- benzoxazole-3-carboxamide |
| 42 | 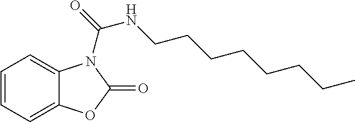 [SMILES: CCCCCCCCNC(=O)n1c(=O)oc2ccccc21] | C₁₆H₂₂N₂O₃ | 290.4 | N-octyl-2-oxo-1,3- benzoxazole-3-carboxamide |
| 43 | 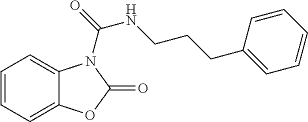 [SMILES: O=C(NCCCc1ccccc1)n1c(=O)oc2ccccc21] | C₁₇H₁₆N₂O₃ | 296.3 | 2-oxo-N-(3-phenylpropyl)-1,3- benzoxazole-3-carboxamide |
| 44 | 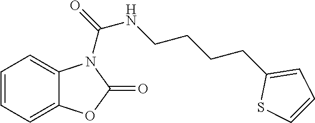 [SMILES: O=C(NCCCCc1cccs1)n1c(=O)oc2ccccc21] | C₁₆H₁₆N₂O₃S | 316.4 | 2-oxo-N-[4-(2-thienyl)butyl]- 1,3-benzoxazole-3- carboxamide |
| 45 | 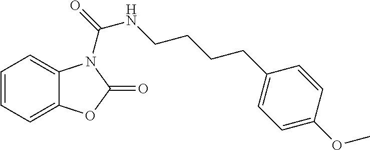 [SMILES: COc1ccc(CCCCNC(=O)n2c(=O)oc3ccccc32)cc1] | C₁₉H₂₀N₂O₄ | 340.4 | N-[4-(4- methoxyphenyl)butyl]-2-oxo- 1,3-benzoxazole-3- carboxamide |
| 46 | 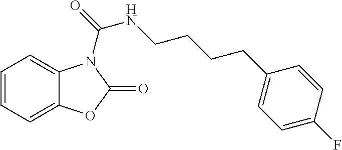 [SMILES: O=C(NCCCCc1ccc(F)cc1)n1c(=O)oc2ccccc21] | C₁₈H₁₇FN₂O₃ | 328.3 | N-[4-(4-fluorophenyl)butyl]-2- oxo-1,3-benzoxazole-3- carboxamide |
| 47 | 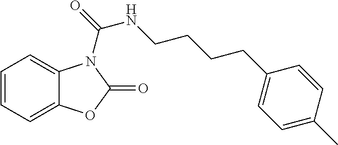 [SMILES: Cc1ccc(CCCCNC(=O)n2c(=O)oc3ccccc32)cc1] | C₁₉H₂₀N₂O₃ | 324.4 | 2-oxo-N-[4-(p-tolyl)butyl]1,3- benzoxazole-3-carboxamide |
| 48 | 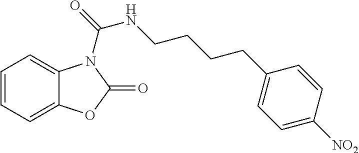 [SMILES: O=C(NCCCCc1ccc([N+](=O)[O-])cc1)n1c(=O)oc2ccccc21] | C₁₈H₁₇N₃O₅ | 355.3 | N-[4-(4-nitrophenyl)butyl]-2- oxo-1,3-benzoxazole-3- carboxamide |
| 49 | 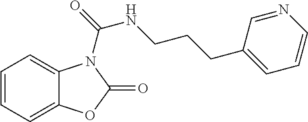 [SMILES: O=C(NCCCc1cccnc1)n1c(=O)oc2ccccc21] | C₁₆H₁₅N₃O₃ | 297.3 | 2-oxo-N-[3-(3-pyridyl)propyl]- 1,3-benzoxazole-3- carboxamide |
| 50 | 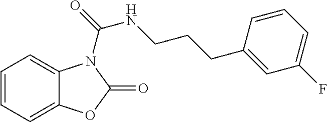 [SMILES: O=C(NCCCc1cccc(F)c1)n1c(=O)oc2ccccc21] | C₁₇H₁₅FN₂O₃ | 314.3 | N-[3-(3-fluorophenyl)propyl]- 2-oxo-1,3-benzoxazole-3- carboxamide |
| 51 | 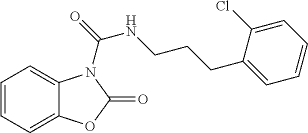 [SMILES: O=C(NCCCc1ccccc1Cl)n1c(=O)oc2ccccc21] | C₁₇H₁₅ClN₂O₃ | 330.8 | N-[3-(2-chlorophenyl)propyl]- 2-oxo-1,3-benzoxazole-3- carboxamide |
| 52 | 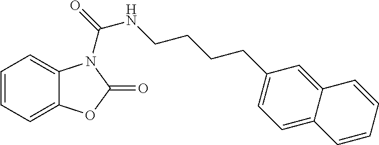 [SMILES: O=C(NCCCCc1ccc2ccccc2c1)n1c(=O)oc2ccccc21] | C₂₂H₂₀N₂O₃ | 360.4 | N-[4-(2-naphthyl)butyl]-2-oxo- 1,3-benzoxazole-3- carboxamide |
| 53 | 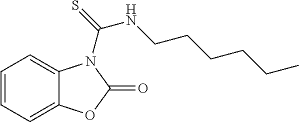 [SMILES: CCCCCCNC(=S)n1c(=O)oc2ccccc21] | C₁₄H₁₈N₂O₂S | 278.4 | N-hexyl-2-oxo-1,3- benzoxazole-3-carbothioamide |
| 54 | 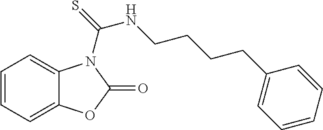 [SMILES: O=c1oc2ccccc2n1C(=S)NCCCCc1ccccc1] | C₁₈H₁₈N₂O₂S | 326.4 | 2-oxo-N-(4-phenylbutyl)-1,3- benzoxazole-3-carbothioamide |
| 55 | 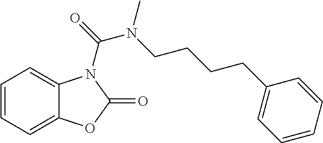 [SMILES: CN(CCCCc1ccccc1)C(=O)n1c(=O)oc2ccccc21] | C₁₉H₂₀N₂O₃ | 324.4 | N-methyl-2-oxo-N-(4- phenylbutyl)-1,3-benzoxazole- 3-carboxamide |
| 56 | 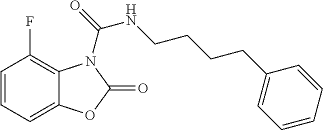 [SMILES: O=C(NCCCCc1ccccc1)n1c(=O)oc2cccc(F)c21] | C₁₈H₁₇FN₂O₃ | 328.3 | 4-fluoro-2-oxo-N-(4- phenylbutyl)-1,3-benzoxazole- 3-carboxamide |
| 57 | 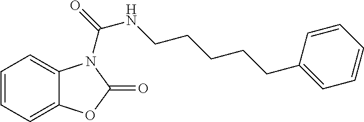 [SMILES: O=C(NCCCCCc1ccccc1)n1c(=O)oc2ccccc21] | C₁₉H₂₀N₂O₃ | 324.4 | 2-oxo-N-(5-phenylpentyl)-1,3- benzoxazole-3-carboxamide |
| 58 | 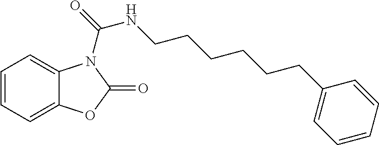 [SMILES: O=C(NCCCCCCc1ccccc1)n1c(=O)oc2ccccc21] | C₂₀H₂₂N₂O₃ | 338.4 | 2-oxo-N-(6-phenylhexyl)-1,3- benzoxazole-3-carboxamide |
| 59 | 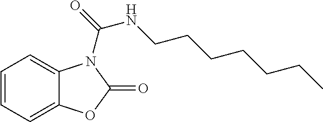 [SMILES: CCCCCCCNC(=O)n1c(=O)oc2ccccc21] | C₁₅H₂₀N₂O₃ | 276.3 | N-heptyl-2-oxo-1,3- benzoxazole-3-carboxamide |

In one embodiment, an exemplary acid ceramidase inhibitor is a compound of Formula IV:

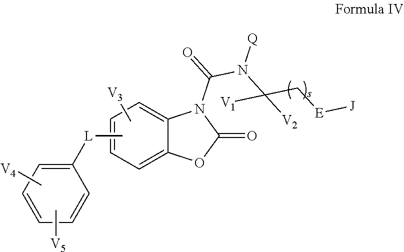

or a pharmaceutically acceptable salt thereof, wherein:

L is a bond, CO, CH(OH) or CH₂;

L can be attached to any position of the ring to which it connected,

Q, V₁ and V₂ are independently hydrogen, linear or branched C₁₋₆ alkyl;

s is an integer from 1 to 6;

J is a linear or branched C₁₋₉ alkyl, C₂₋₉ alkenyl or C₂₋₉ alkynyl group or a group:

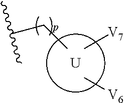

wherein:

p is 0 or an integer from 1 to 6;

U is a 3-10 membered saturated, unsaturated, aromatic or heteroaromatic, single or fused ring comprising up to three heteroatoms selected from N, O, S; and V₆ and V₇ are as defined below;

V₃ is hydrogen, halogen, linear or branched C₁₋₆ alkyl, C₁₋₆ alkoxy or OH;

V₃ can be attached to any position of the ring to which it is connected;

V₄ and V₅ are independently selected from the group consisting of hydrogen, halogen, linear or branched C₁₋₆ alkyl, C₁₋₆ alkoxy, hydroxy C₁₋₆ alkyl, OH, CN, NO₂, fluoro C₁₋₆ alkyl, fluoro C₁₋₆ alkoxy, COOV₈, CONV₉V₁₀, SO₂NV₉V₁₀, SO₂V₁₁;

V₆ and V₇ are independently selected from the group consisting of hydrogen, halogen, linear or branched C₁₋₆ alkyl, optionally substituted C₃₋₆ cycloalkyl, C₁₋₆ alkoxy, hydroxy C₁₋₆ alkyl, OH, CN, NO₂, fluoro C₁₋₆ alkyl, fluoro C₁₋₆ alkoxy, optionally substituted aryl or heteroaryl, COOV₈, CONV₉V₁₀, SO₂NV₉V₁₀, SO₂V₁₁;

V₄, V₅, V₆ and V₇ can be attached to any position of the ring to which they are connected;

E is a bond or a heteroatom selected from the group consisting of O, S, SO, SO₂ or NV₁₂;

V₈, V₉, V₁₀, V₁₁ and V₁₂ are independently selected from the group consisting of hydrogen, linear or branched C₁₋₆ alkyl;

provided that when E is a bond, both the following conditions are met:

J is a group:

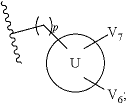

and s+p is >4.

In certain embodiments, compounds of Formula (IV) as defined above are provided wherein:

L is a bond, CO, CH(OH);

Q Is hydrogen;

V₁ and V₂ are independently hydrogen, linear or branched C₁₋₆ alkyl, preferably methyl;

s is an integer from 1 to 6;

J is a linear C₁₋₆ alkyl or a group:

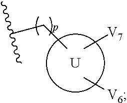

p is an integer from 1 to 6;

U is an aryl selected from naphthyl or phenyl, (C₃-C₁₀)cycloalkyl, or a heteroaryl which is pyridyl, thiophenyl, pyrimidinyl, furyl, or indolyl;

V₃ is hydrogen, halogen, preferably chlorine or fluorine;

V₄ and V₅ are independently selected from the group consisting of hydrogen, halogen preferably F, linear or branched C₁₋₆ alkyl preferably C₁₋₃ alkyl, C₁₋₆ alkoxy preferably MeO and EtO, OH, CN, NO₂, CF₃, hydroxy C₁₋₆ alkyl;

V₆ and V₇ are independently selected from the group consisting of hydrogen, halogen, linear or branched C₁₋₆ alkyl, C₁₋₆ alkoxy, preferably MeO and EtO, hydroxy C₁₋₆ alkyl, OH, CN, NO₂, CF₃; preferably both V₆ and V₇ are hydrogen;

E is a bond or a heteroatom selected from the group consisting of O, S, SO, SO₂;

with the proviso that when E is a bond, J is a group:

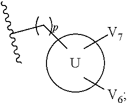

and s+p is >4.

Exemplary compounds of Formula IV are set forth in Table 2.

TABLE 2
Exemplary compounds of Formula IV

| Example | Structure | Formula | MW | Name |
|---|---|---|---|---|
| 1 | 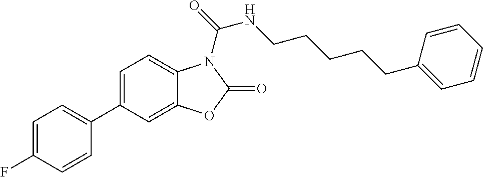 [SMILES: O=C(NCCCCCc1ccccc1)n1c(=O)oc2cc(-c3ccc(F)cc3)ccc21] | C₂₅H₂₃FN₂O₃ | 418.5 | 6-(4-fluorophenyl)-2-oxo-N-(5- phenylpentyl)-1,3-benzoxazole- 3-carboxamide |
| 2 | 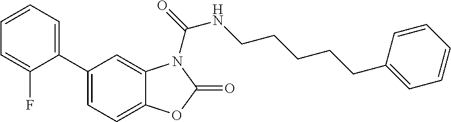 [SMILES: O=C(NCCCCCc1ccccc1)n1c(=O)oc2ccc(-c3ccccc3F)cc21] | C₂₅H₂₃FN₂O₃ | 418.5 | 6-(2-fluorophenyl)-2-oxo-N-(5- phenylpentyl)-1,3-benzoxazole- 3-carboxamide |
| 3 | 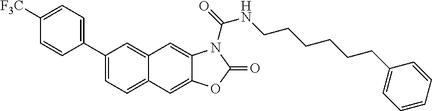 [SMILES: O=C(NCCCCCCc1ccccc1)n1c(=O)oc2cc3ccc(-c4ccc(C(F)(F)F)cc4)cc3cc21] | C₂₇H₂₅F₃N₂O₃ | 482.5 | 2-oxo-N-(6-phenylhexyl)-5-[4- (trifluoromethyl)phenyl]-1,3- benzoxazole-3-carboxamide |
| 4 | 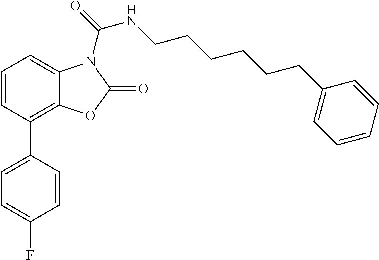 [SMILES: O=C(NCCCCCCc1ccccc1)n1c(=O)oc2c(-c3ccc(F)cc3)cccc21] | C₂₆H₂₅FN₂O₃ | 432.5 | 7-(4-fluorophenyl)-2-oxo-N-(6- phenylhexyl)-1,3-benzoxazole-3- carboxamide |
| 5 | 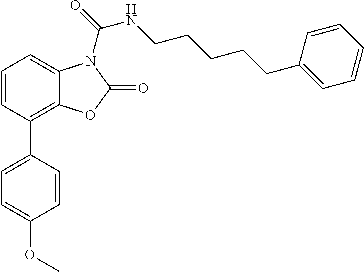 [SMILES: COc1ccc(-c2cccc3c2oc(=O)n3C(=O)NCCCCCc2ccccc2)cc1] | C₂₆H₂₆N₂O₄ | 430.5 | 7-(4-methoxyphenyl)-2-oxo-N- (5-phenylpentyl)-1,3- benzoxazole-3-carboxamide |
| 6 | 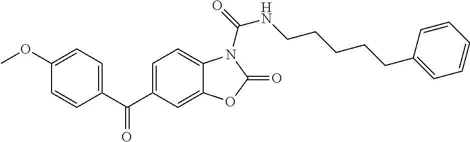 [SMILES: COc1ccc(C(=O)c2ccc3c(c2)oc(=O)n3C(=O)NCCCCCc2ccccc2)cc1] | C₂₇H₂₆N₂O₅ | 458.5 | 6-(4-methoxyphenyl)-2-oxo-N- (5-phenylpentyl)-1,3- benzoxazole-3-carboxamide |
| 7 | 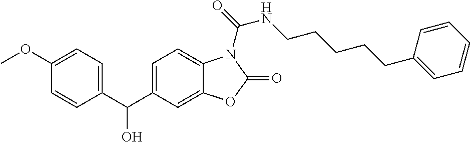 [SMILES: COc1ccc(C(O)c2ccc3c(c2)oc(=O)n3C(=O)NCCCCCc2ccccc2)cc1] | C₂₇H₂₈N₂O₅ | 460.5 | (±)-6-[hydroxyl-(4- methoxyphenyl)methyl]-2-oxo- N-(5-phenylpentyl)-1,3- benzoxazole-3-carboxamide |
| 8 | 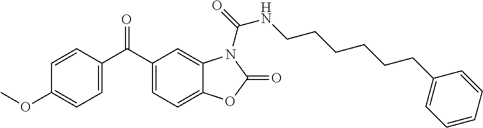 [SMILES: COc1ccc(C(=O)c2ccc3oc(=O)n(C(=O)NCCCCCCc4ccccc4)c3c2)cc1] | C₂₆H₂₈N₂O₅ | 472.5 | 5-(4-methoxybenzoyl)-2-oxo-N- (6-phenylhexyl)-1,3- benzoxazole-3-carboxamide |
| 9 | 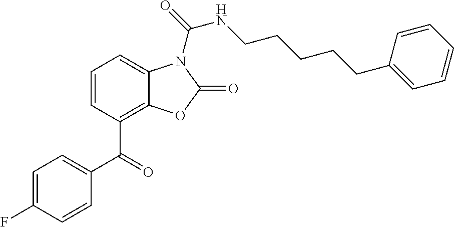 [SMILES: O=C(c1ccc(F)cc1)c1cccc2c1oc(=O)n2C(=O)NCCCCCc1ccccc1] | C₂₆H₂₃FN₂O₄ | 446.5 | 7-(4-fluorobenzoyl)-2-oxo-N-(5- phenylpentyl)-1,3-benzoxazole- 3-carboxamide |
| 10 | 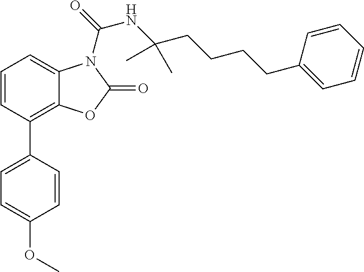 [SMILES: COc1ccc(-c2cccc3c2oc(=O)n3C(=O)NC(C)(C)CCCCc2ccccc2)cc1] | C₂₈H₃₀N₂O₄ | 458.6 | N-(1,1-dimethyl-5-phenyl- pentyl)-7-(4-methoxyphenyl)-2- oxo-1,3-benzoxazole-3- carboxamide |
| 11 | 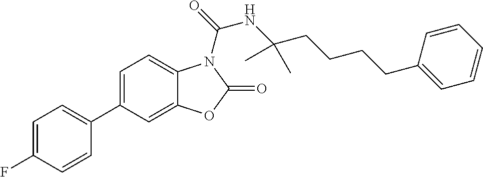 [SMILES: CC(C)(CCCCc1ccccc1)NC(=O)n1c(=O)oc2cc(-c3ccc(F)cc3)ccc21] | C₂₇H₂₇FN₂O₃ | 446.5 | N-(1,1-dimethyl-5-phenyl- pentyl)-6-(4-fluorophenyl)-2- oxo-1,3-benzoxazole-3- carboxamide |
| 12 | 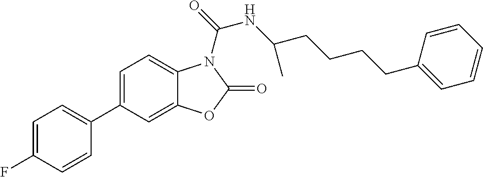 [SMILES: CC(CCCCc1ccccc1)NC(=O)n1c(=O)oc2cc(-c3ccc(F)cc3)ccc21] | C₂₆H₂₅FN₂O₃ | 432.5 | (±)-6-(4-fluorophenyl)-N-(1- methyl-5-phenyl-pentyl)-2-oxo- 1,3-benzoxazole-3-carboxamide |
| 13 | 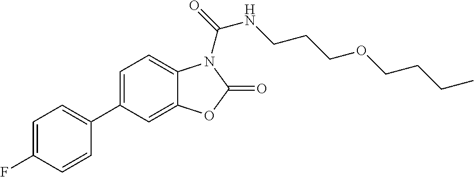 [SMILES: CCCCOCCCNC(=O)n1c(=O)oc2cc(-c3ccc(F)cc3)ccc21] | C₂₁H₂₃FN₂O₄ | 386.4 | N-(3-butoxypropyl)-6-(4- fluorophenyl)-2-oxo-1,3- benzoxazole-3-carboxamide |
| 14 | 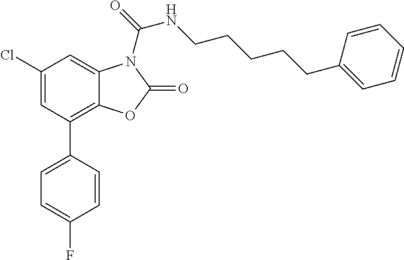 [SMILES: O=C(NCCCCCc1ccccc1)n1c(=O)oc2c(-c3ccc(F)cc3)cc(Cl)cc21] | C₂₅H₂₂ClFN₂O₃ | 451.9 | 5-chloro-7-(4-fluorophenyl)-2- oxo-N-(5-phenylpentyl)-1,3- benzoxazole-3-carboxamide |
| 15 | 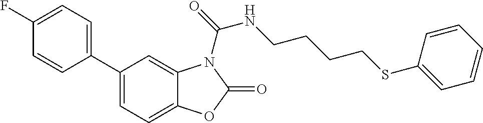 [SMILES: O=C(NCCCCSc1ccccc1)n1c(=O)oc2ccc(-c3ccc(F)cc3)cc21] | C₂₄H₂₁FN₂O₃S | 436.5 | 5-(4-fluorophenyl)-2-oxo-N-(4- phenylsulfanylbutyl)-1,3- benzoxazole-3-carboxamide |
| 16 | 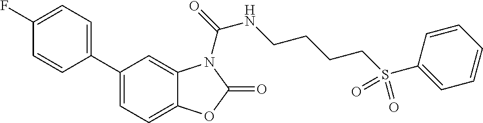 [SMILES: O=C(NCCCCS(=O)(=O)c1ccccc1)n1c(=O)oc2ccc(-c3ccc(F)cc3)cc21] | C₂₄H₂₁FN₂O₅S | 468.5 | N-[4-(benzenesulfonyl)butyl]-5- (4-fluorophenyl)-2-oxo-1,3- benzoxazole-3-carboxamide |
| 17 | 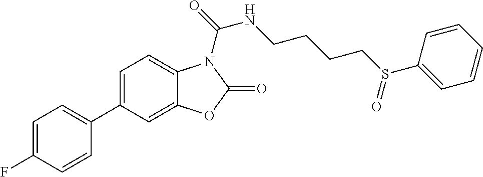 [SMILES: O=C(NCCCCS(=O)c1ccccc1)n1c(=O)oc2cc(-c3ccc(F)cc3)ccc21] | C₂₄H₂₁FN₂O₄S | 452.5 | (±)-N-[4-(benzenesulfinyl)butyl]- 6-(4-fluorophenyl)-2-oxo-1,3- benzoxazole-3-carboxamide |

### Combination Therapy

The invention embraces combination therapy, which includes the administration of an acid ceramidase inhibitor and a second agent as part of a specific treatment regimen intended to provide the beneficial effect from the co-action of these therapeutic agents. The beneficial effect of the combination may include pharmacokinetic or pharmacodynamic co-action resulting from the combination of therapeutic agents.

For example, during the treatment of Parkinson's disease, the acid ceramidase inhibitor can be administered in combination with carbidopa and/or levadopa, a dopamine agonist, a monoamine oxidase B inhibitor, a catchetol O-methyltransferase inhibitor, an anticholingeric, or amantadine. During the treatment of Alzheimer's disease, the acid ceramidase inhibitor can be administered in combination with a cholinesterase inhibitor and/or memantine. During the treatment of Huntington's disease, the acid ceramidase inhibitor can be administered in combination with tetrabenazine; an antipsychotic drug such as haloperidol, chlorpromazine, quetiapine, risperidone, and olanzapine; a chorea-suppressing medication such as amantadine, levetiracetam, and clonazempam; an antidepressant such as citalopram, fluoxetine, and sertraline; and a mood-stabilizing drug such as valproate, carbamazepine, and lamotrigine. During the treatment of amyotrophic lateral sclerosis, the acid ceramidase inhibitor can be administered in combination with riluzole; an agent for ameliorating muscle cramps and spasms such as cyclobenzaprine HCL, metaxalone, and robaxin; an agent for ameliorating spasticity such as tizanidine HCl, baclofen, and dantrolene; an agent for ameliorating constipation such as lubiprostone, linaclotide, lactulose, and polyethylene glycol; an agent for ameliorating fatigue such as caffeine, caffeine citrate, or caffeine benxoate injection; an agent for ameliorating excessive salivation such as glycopyrrolate, propantheline, amitriptyline, nortriplyline HCL and scopolamine; an agent for ameliorating excessive phlegm such as guaifenesin, albuterol inhalation, and acetylcysteine; an agent for ameliorating pain such as an opioid; an anticonvulsant or antiepileptic; a serotonin reuptake inhibitor, an antidepressant; an agent for ameliorating sleep disorders such as a benzodiazepine, a non-benzodiazepine hypnotic, a melatonin receptor stimulator, an anti-narcoleptic, and an orexin receptor antagonist; and an agent pseudobulbar affect such as dextromethorphan/quinidine. During the treatment of multiple sclerosis, the acid ceramidase inhibitor can be administered in combination with a corticosteroid, P interferon, glatiramer acetate, dimethyl fumarate, fingolimod, teriflunomide, natalizumab, mitoxantrone, baclofen, and tizanidine. During the treatment of diffuse Lewy body disease, the acid ceramidase inhibitor can be administered in combination with a cholinesterase inhibitor, a Parkinson's disease medication such as carbidopa and/or levodopa, and an anti-psychotic medication such as quetiapine and olanzapine. During the treatment of multisystem atrophy, the acid ceramidase inhibitor can be administered in combination with a medication to raise blood pressure such as fludrocortisone, psyridostigmine, midodrine, and droxidopa; and a Parkinson's disease medication such as carbidopa and/or levodopa. During the treatment of frontotemporal dementia, the acid ceramidase inhibitor can be administered in combination with an antidepressant, a selective serotonin reuptake inhibitor, and an antipsychotic. During the treatment of progressive upranuclear palsy, the acid ceramidase inhibitor can be administered in combination with a Parkinson's disease medication such as carbidopa and/or levodopa. It is understood that other combinations would be known be those skilled in the art.

### IV. Pharmaceutical Compositions

The acid ceramidase inhibitors described hereinabove useful in the treatment of neurodegenerative disorders can be present in a pharmaceutical composition. In certain embodiments, the pharmaceutical compositions preferably comprise a therapeutically-effective amount of one or more of the acid ceramidase inhibitors described above formulated together with one or more pharmaceutically acceptable carriers. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets (e.g., those targeted for buccal, sublingual, and/or systemic absorption), boluses, powders, granules, pastes for application to the tongue; (2) parenteral administration by, for example, subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration.

The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-Ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffin's, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

When the acid ceramidase inhibitor is delivered by systemic administration, the inhibitor preferably is capable of traversing the blood-brain barrier. Alternatively, the inhibitor may be formulated using formulation techniques know in the art for enhancing traversal of an active agent across the blood-brain barrier. For example, the acid ceramidase inhibitor may be co-administered with an agent that transiently increases the permeability of the blood-brain barrier, including, for example, bradykinin, or Cereport, a nine amino acid peptide based on bradykinin (Alkermes, Cambridge, Mass.). Alternatively, or in addition, the acid ceramidase inhibitor may be administered following a procedure that transiently increases the permeability of the blood-brain barrier, including, for example, localized exposure to high-intensity focused ultrasound, and osmotic disruption of the blood-brain barrier through induced shrinkage of cerebrovascular endothelial cells. In addition, exemplary approaches for formulating the acid ceramidase inhibitor to facilitate transport across the blood-brain barrier, which can be used either alone or in combination with a method and/or composition for transiently increasing permeability of the blood-brain barrier, include encapsulation in a particle capable of traversing the blood-brain barrier, including, for example, lipid nanoparticles (Tekmira, British Columbia, Canada); liposomes (2-BBB, Leiden, Netherlands); chitosen nanoparticles; dendrimers; poly (D,L-lactide-co-glycolide) nanoparticles; poly (D,L-lactide) nanoparticles; and polybutylcyanoacrylate nanoparticles. For a review of different approaches for enhancing delivery of a pharmacologically active agent across the blood-brain carrier, see, Pardridge (2012) J. CEREBRAL BLOOD FLOW & METABOLISM 32: 1959-1972.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factor well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved. In other words, a compound of the invention may be tritrated by a physician or veterinarian at escalating dosages to the subject over a period of days, weeks, or months to ameliorate at least an symptom associated with the neurodegenerative disorder in question, including loss of cognitive function and/or cognitive impairment.

In general, a suitable daily dose of a compound of the invention will be that amount of the compound which is the lowest dose effective to produce a therapeutic effect. Such an effective dose will generally depend upon the factors described above. The compound or compounds can administered at about 0.01 mg/kg to about 200 mg/kg, or about 0.1 mg/kg to about 100 mg/kg, or at about 0.5 mg/kg to about 50 mg/kg. In certain embodiments, the compound or compounds can be administered at a concentration less than 20 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

Under certain circumstances, the acid ceramidase inhibitor, when administered to a subject, does not result in complete inhibition of the target acid ceramidase activity. Rather the amount of the acid ceramidase inhibitor is titrated to permit the target ceramidase to synthesize a sufficient amount of the sphingosine-containing analog for normal cellular function. In other words, the acid ceramidase inhibitor preferentially prevents an accumulation of the sphingosine-containing analog to abnormal levels, which become detrimental to cells and cellular function. The ceramidase inhibitor preferably reduces activity of the target ceramidase in a cell or tissue sample by less than 80%, 70%, 60%, 50%, 40%, 30%, 20%, 10% or 5% relative to activity prior to exposure by the inhibitor as determined by an n vitro assay, such as a fluorogenic assay employing a fluorogenic substrate, for example, Rbm14-12 (Bedia et al. (2010) supra). Furthermore, the acid ceramidase inhibitor should be titrated to permit the conversion of ceramide to sphingosine to provide normal or substantially normal levels of sphingosine in the subject. This can be accomplished by titrating the dosage of the inhibitor to establish the appropriate inhibition of acid ceramidase activity in the subject. This can be accomplished by employing a fluorogenic assay, for example, a fluorogenic assay using the fluorogenic substrate Rbm14-12 (Bedia et al., supra) to measure ceramidase activity in peripheral blood mononuclear cells extracted from the subject.

### V. Kits for Use in Medical Applications

Another aspect of the invention provides a kit for treating a neurodegenerative disorder. The kit comprises: (i) instructions for treating a medical disorder, such as Parkinson's disease; and (ii) an acid ceramidase inhibitor. The kit may comprise one or more unit dosage forms containing an amount of an acid ceramidase inhibitor that is effective for treating the neurodegenerative disorder, for example, Parkinson's disease.

### EXAMPLE

### Example 1: Evaluation of Acid Ceramidase Inhibitors for Use in Treatment of Neurodegenerative Disorders

This example describes the acid ceramidase inhibition activity of compound 25 of Table I (Formula III).

Inhibition of acid ceramidase activity by compound 25 of Table 1 was evaluated in a fluorescent intensity assay using a fluorogenic substrate Rbm 14-12 (RNA-binding protein 14-12). Compound 25 was incubated with cell lysates enriched with acid ceramidase for 1 hour in an assay buffer containing 50 mM NaOAc and 100 mM NaCl at pH 4.5. The reaction was initiated by the addition of the substrate at a final concentration of 6.3 μM and the mixture was incubated at room temperature for 1 or two hours. At the appropriate time, the reaction was quenched by the addition of methanol and treated with NaIO₄ (fresh 2.5 mg/ml solution was made in 100 mM glycine/NaOH buffer, pH 10.6), followed by incubation for 1 hour at room temperature. Fluorescent intensity was measured using a plate reader at ex 355 nm and em 460 nm. The obtained average IC₅₀ value for the 1 and 2 hour time points was in the range of 250 nM to 500 nM.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A method of treating a neurodegenerative disorder in a subject in need thereof the method comprising administering to the subject an acid ceramidase inhibitor in an amount effective to treat the disorder in the subject.

2. The method of claim 1, wherein said disorder is Parkinson's disease, Alzheimer's Disease, Huntington's Disease, amyotrophic lateral sclerosis, multiple sclerosis, diffuse Lewy body disease, multisystem atrophy, frontotemporal dementia, or progressive supranuclear palsy.

3. The method of claim 1 or 2, wherein the acid inhibitor prevents the accumulation of a glycosphingosine to a level found in subjects with the neurodegenerative disorder when compared to subjects without the disorder.

4. The method of any one of claims 1-3, wherein the acid ceramide inhibitor is a compound of Formula I or Formula I-1:
(a) Formula I: 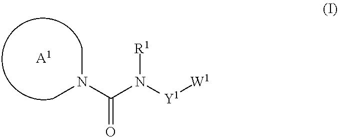
or a pharmaceutically acceptable salt thereof, wherein:
A¹ is a cyclic group selected from 5-6 membered heterocyclyl, 5-6 membered heteroaryl, and bicyclic heterocyclyl, each of which is substituted by 1, 2, 3, or 4 occurrences of R²;
R¹ represents independently for each occurrence hydrogen, C₁₋₄alkyl, —C₁₋₄alkyl-phenyl, —CO₂—C₁₋₆alkyl, —C(O)—NH₂, —C(O)—NH—C₁₋₆alkyl, or —C(O)—N(C₁₋₆alkyl)₂;
R² represents independently for each occurrence R¹, C₁₋₄alkyl, C₁₋₄haloalkyl, C₁₋₄alkoxy, halogen, hydroxyl, oxo, cyano, nitro, azido, —N(R¹)₂, —C(O)—C₁₋₄alkyl, —C(O)-phenyl, —CO₂—R¹, —C(O)—NH₂, —C(O)—NH—C₁₋₆alkyl, —C(O)—N(C₁₋₆alkyl)₂, —O—C(O)—NH₂, —O—C(O)—NH—C₁₋₆alkyl, —O—C(O)—N(C₁₋₆alkyl)₂, —C₁₋₄alkyl-phenyl, C₃₋₁₀cycloalkyl, C₃₋₁₀heterocyclyl, 6-10 membered aryl, 6-10 membered heteroaryl, —C₁₋₄alkylene-C₃₋₁₀cycloalkyl, —C₁₋₄alkylene-C₃₋₁₀heterocyclyl, —(C₁₋₄alkylene)-6-10 membered aryl, or —(C₁₋₄alkylene)-6-10 membered heteroaryl;
Y¹ represents:
C₁₋₁₈alkylene, C₂₋₁₈alkenylene, or C₂₋₁₈alkynylene;
C₃₋₁₀cycloalkylene, 3-10 membered heterocyclylene, 6-10 membered arylene, or 6-10 membered heteroarylene, each of which is substituted by 0, 1, 2, or 3 occurrences of C₁₋₄alkyl; or
R¹ and Y¹ together with the nitrogen to which they are attached form a 3-10 membered heterocyclylene; and
W¹ represents:
hydrogen; or
C₃₋₁₀cycloalkylene, C₃₋₁₀heterocyclylene, 6-10 membered arylene, or 6-10 membered heteroarylene,
or (b) Formula I-1: 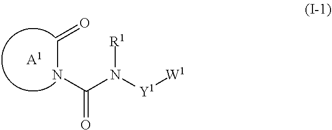
or a pharmaceutically acceptable salt thereof, wherein:
A¹ is a cyclic group selected from 5-6 membered heterocyclyl, 5-6 membered heteroaryl, and bicyclic heterocyclyl, each of which is substituted by 1, 2, or 3 occurrences of R²;
R¹ represents independently for each occurrence hydrogen, C₁₋₄alkyl, —C₁₋₄alkyl-phenyl, —CO₂C₁₋₆alkyl, —C(O)—NH₂, —C(O)—NH—C₁₋₆alkyl, or —C(O)—N(C₁₋₆alkyl)₂;
R² represents independently for each occurrence R¹, C₁₋₄alkyl, C₁₋₄haloalkyl, C₁₋₄alkoxy, halogen, hydroxyl, oxo, cyano, nitro, azido, —N(R¹)₂, —C(O)—C₁₋₄alkyl, —C(O)-phenyl, —CO₂—R¹, —C(O)—NH₂, —C(O)—NH—C₁₋₆alkyl, —C(O)N(C₁₋₆alkyl)₂, —O—C(O)—NH₂, —O—C(O)—NH—C₁₋₆alkyl, —O—C(O)—N(C₁₋₆alkyl)₂, —C₁₋₄alkyl-phenyl, C₃₋₁₀cycloalkyl, C₃₋₁₀heterocyclyl, 6-10 membered aryl, 6-10 membered heteroaryl, —C₁₋₄alkylene-C₃₋₁₀cycloakyl, —C₁₋₄alkylene-C₃₋₁₀heterocyclyl, —C₁₋₄alkylene-6-10 membered aryl, or —C₁₋₄alkylene-6-10 membered heteroaryl;
Y¹ represents:
C₁₋₁₈alkylene, C₂₋₁₈alkenylene, or C₂₋₁₈alkynylene;
C₃₋₁₀cycloalkylene, 3-10 membered, 6-10 membered arylene, or 6-10 membered heteroarylene, each of which is substituted by 0, 1, 2, or 3 occurrences of C₁₋₄alkyl; or
R¹ and Y¹ together with the nitrogen to which they are attached form a 3-10 membered heterocyclylene; and
W¹ represents:
hydrogen; or
C₃₋₁₀cycloalkylene, C₃₋₁₀heterocyclylene, 6-10 membered arylene, or 6-10 membered heteroarylene.

5. The method of any one of claims 1-4, wherein the inhibitor is selected from the group consisting of: 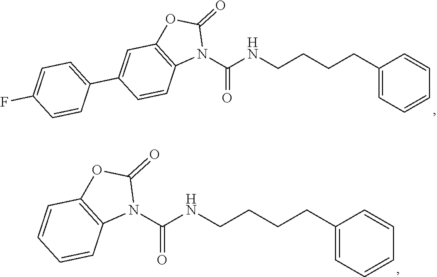 [SMILES: O=C(NCCCCc1ccccc1)n1c(=O)oc2cc(-c3ccc(F)cc3)ccc21 ; O=C(NCCCCc1ccccc1)n1c(=O)oc2ccccc21]
pharmaceutically acceptable salts thereof.

6. The method of any one of claims 1-4, wherein the inhibitor is a uracil analog.

7. The method of claim 6, wherein the inhibitor is a 5-fluorouracil analog.

8. The method of claim 7, wherein the inhibitor is 1-hexylcarbamoyl-5-fluorouracil

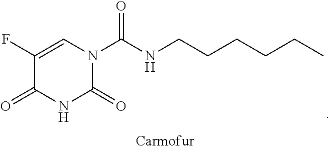 [SMILES: CCCCCCNC(=O)n1cc(F)c(=O)[nH]c1=O]

9. The method of claim 7 or 8, wherein the acid ceramidase inhibitor is administered at a concentration sufficient to inhibit acid ceramidase activity without substantially inhibiting thymidylate synthase activity.

10. The method of any one of claims 1-9, wherein the acid ceramidase inhibitor is administered at a concentration less than 20 mg/kg.
